(19) United States
(12) Patent Application Publication
(10) Pub. No.: US 2024/0307395 A1
(43) Pub. Date: Sep. 19, 2024

(54) USE OF BI853520 IN CANCER TREATMENT

(71) Applicant: INXMED (NANJING) CO., LTD., Nanjing (CN)

(72) Inventors: Zaiqi Wang, Nanjing (CN); Jiangwei Zhang, Shanghai (CN); Jun Jiang, Beijing (CN); Baoyuan Zhang, Shanghai (CN); Xuebin Liu, Nanjing (CN)

(21) Appl. No.: 18/674,295

(22) Filed: May 24, 2024

Related U.S. Application Data
(63) Continuation-in-part of application No. 18/221,588, filed on Jul. 13, 2023.
Application No. 18/221,588 is a continuation of application No. 17/780,397, filed on May 26, 2022.

(30) Foreign Application Priority Data
Nov. 28, 2019 (CN) 201911191139.8
Nov. 17, 2020 (CN) 202011287049.1

(51) Int. Cl. A61K 31/513; A61K 31/519; A61P 35/00

(52) U.S. Cl. CPC A61K 31/513; A61K 31/519; A61P 35/00

(57) ABSTRACT

The present invention relates to a method for treating a tumor such as a tumor with a KRAS mutation. In the method, a compound 2-fluoro-5-methoxy-4-[(4-(2-methyl-3-oxo-2,3-dihydro-1H-isoindole-4-oxy)-5-trifluoromethyl-pyrimidine-2-yl)amino]-N-(1-methyl-piperidine-4-yl)benzamide or a pharmaceutically acceptable salt thereof is used, which can be administered alone or in combination with a KRAS inhibitor and/or an MEK inhibitor.

### CROSS REFERENCE TO RELATED APPLICATIONS

This application is a continuation-in-part application of U.S. patent application Ser. No. 18/221,588, filed Jul. 13, 2023, which is a continuation of U.S. patent application Ser. No. 17/780,397 filed May 26, 2022, which is a National Stage application under 35 C.F.R. § 371 of PCT/CN2020/132244, filed Nov. 27, 2020, which claims the priority of the Chinese Patent Application No. 201911191139.8 filed on Nov. 28, 2019 and the Chinese Patent Application No. 202011287049.1 filed on Nov. 17, 2019, the contents of which are incorporated herein by reference as a part of this application.

### FIELD OF THE INVENTION

The present disclosure belongs to the field of pharmaceutical chemistry, and particularly relates to use of a compound in the manufacture of a medicament for treating a tumor such as a tumor with a KRAS mutation.

### BACKGROUND OF THE INVENTION

The Ras gene family encodes small GTPases involved in cell signaling. Mutations in the Ras gene can leave it permanently activated and lead to inappropriate intracellular signaling in the absence of extracellular signals. Because signaling leads to cell growth and division, dysregulated Ras signaling may ultimately lead to tumorigenesis and cancer. The Ras gene encodes the Ras superfamily proteins, which include the KRAS (Kirsten rat sarcoma viral oncogene homolog) protein encoded by the KRAS gene.

KRAS gene mutations are common in pancreatic, lung, colorectal, gallbladder, thyroid, and bile duct cancers. Epidermal growth factor receptor (EGFR) TKIs are commonly used to treat these cancers. It has been reported that KRAS mutation is a predictor of the tumor's response to epidermal growth factor receptor (EGFR) TKI targeted therapy. The most common KRAS mutations occur at codons 12 and 13 of exon 2. Other rare mutations occur at codons 59 and 61 of exon 3. Studies found that KRAS mutations at codons 12, 13 or 61 cause the Ras protein to remain in its active form for a longer time, leading to overactivation of the EGFR pathway. Therefore, patients with a KRAS mutation at codons 12, 13, or 61 do not respond well to tyrosine kinase inhibitor therapy. Furthermore, it has been shown that a KRAS mutation at codon 12 or 13 is a strong predictor of patients who are in non-response to anti-EGFR monoclonal antibody therapy such as for the treatment of certain cancerous diseases, including ERBITUX® (cetuximab; ImClone Systems Inc., New York, USA) and VECTIBIX® (panitumumab, Amgen, Thousand Oaks, CA, USA) for metastatic colorectal cancer (mCRC) and lung cancer. See Massarelli et al., KRAS Mutation is an Important Predictor of Resistance to Therapy with Epidermal Growth Factor Receptor Tyrosine Kinase Inhibitors in Non-Small Cell Lung Cancer, CLIN CANCER RES., 13(10):2890-2896 (2007); Amado et al., Wild-type KRAS is Required for Panitumumab Efficiency in Patients with Metastatic Colorectal Cancer, J. CLIN ONCOL, 26(10):1626-1634 (2008); Van Cutsem et al., KRAS Status and Efficacy in the First-Line Treatment of Patients with Metastatic Colorectal Cancer (mCRC) Treated with FOLFIRI with or without Cetuximab: The CRYSTAL Experience, J CLIN ONCOL, 26(15S): May 20 Supplement, Abstract 2 (2008); Baker et al., Evaluation of Tumor Gene Expression and KRAS Mutations in FFPE Tumor Tissue as Predictors of Response to Cetuximab in Metastatic Colorectal Cancer, J CLIN ONCOL, 26(15S): May 20 Supplement, Abstract 3512 (2008); Van Zakowski et al., Reflex Testing of Lung Adenocarcinomas for EGFR and KRAS Mutations: The Memorial Sloan-Kettering Experience, J. CLIN ONCOL, 26(15S): May 20 Supplement, Abstract 22031 (2008).

Therefore, there is a need to find a therapy for cancers with a KRAS mutation.

### SUMMARY OF THE INVENTION

In one aspect, the present disclosure provides use of a compound or a pharmaceutically acceptable salt thereof in the manufacture of a medicament for treating a tumor such as a tumor with a KRAS mutation, wherein the compound is 2-fluoro-5-methoxy-4-[(4-(2-methyl-3-oxo-2,3-dihydro-1H-isoindole-4-oxy)-5-trifluoromethyl-pyrimidine-2-yl)amino]-N-(1-methyl-piperidine-4-yl)benzamide (hereinafter referred to as “the compound”, also referred to as “BI853520”, see WO2010058032), wherein the compound has a structure of:

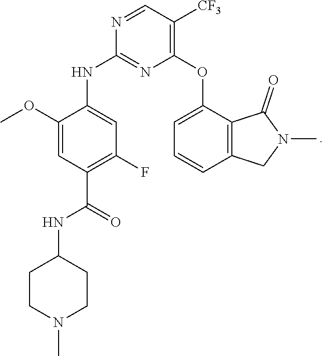

The compound was particularly effective in a tumor with a KRAS mutation (see Example 1). In addition, the compound was found to increase efficacy in combination with a KRAS inhibitor or a mitogen-activated protein kinase (MEK) inhibitor, resulting in durable and sustained tumor regression (see Examples 2 to 11).

Optionally, the tumor is pancreatic cancer, colorectal cancer, lung cancer such as lung adenocarcinoma and non-small cell lung cancer, kidney cancer, gastric cancer such as gastric adenocarcinoma cancer, prostate cancer or ovarian cancer.

Optionally, the KRAS mutation is a G12A, G12C, G12D, G12R, G12S, G12V, G13C, G13D, G13V, Q61K, Q61L, Q61R or Q61H mutation.

Optionally, the KRAS mutation is a G12C, G12D, G13C or Q61K mutation. Optionally, the KRAS mutation is a G12C mutation. Optionally, the KRAS mutation is a G12D mutation.

Optionally, the tumor is 1) lung cancer, colorectal cancer or pancreatic cancer with a KRAS G12C mutation; 2) acute myeloid leukemia with a KRAS G12D, KRAS G12V, KRAS G13D or KRAS Q61H mutation; 3) bladder cancer with a KRAS G12C, KRAS G12D, KRAS G12R, KRAS G12V, KRAS G13D, or KRAS Q61H mutation; 4) breast cancer with a KRAS G12C, or KRAS G12V mutation; 5) cervical cancer with a KRAS G12C, KRAS G12D, KRAS G12V, or KRAS G13D mutation; 6) bile duct cancer with a KRAS G12R, or KRAS Q61K mutation; 7) colorectal cancer with a KRAS G12A, KRAS G12C, KRAS G12D, or KRAS G13D mutation; 8) esophageal cancer with a KRAS G12D mutation; 9) gastric cancer with a KRAS G12C, KRAS G12D, KRAS G12S, KRAS G12V, KRAS G13D, or KRAS Q61H mutation; 10) glioblastoma with a KRAS G12D mutation; 11) liver cancer with a KRAS G12C, KRAS G12D, or KRAS G13D mutation; 12) lung cancer with a KRAS G12A, KRAS G12D, KRAS G12S, KRAS G12V, KRAS G13C, KRAS G13D, KRAS Q61K, or KRAS Q61L mutation; 13) melanoma with a KRAS G12C, KRAS G12D, KRAS G12R, KRAS G13D, KRAS Q61K, KRAS Q61L, or KRAS Q61R mutation; 14) mesothelioma with a KRAS G12C mutation; 15) ovarian cancer with a KRAS G12R, KRAS G12V, KRAS Q61L, or KRAS G13C mutation; 16) pancreatic cancer with a KRAS G12A, KRAS G12D, KRAS G12R, KRAS G12V, KRAS G13C, or KRAS Q61H mutation; 17) prostate cancer with a KRAS G12D, KRAS G12R, or KRAS G12V mutation; 18) kidney cancer with a KRAS G12C, KRAS G12D, or KRAS G12V mutation; 19) sarcoma with a KRAS G13C, or KRAS Q61H mutation; 20) thyroid cancer with a KRAS G12V, KRAS Q61K, or KRAS Q61R mutation; 21) testicular cancer with a KRAS G12A, KRAS G12R, KRAS G12S, KRAS G12V, KRAS Q61L, or KRAS Q61R mutation; 22) thymoma with a KRAS G12D mutation; or 23) metrocarcinoma with a KRAS G12A, KRAS G12C, KRAS G12D, KRAS G12S, KRAS G12V, KRAS G13C, KRAS G13D, KRAS G13V, KRAS Q61H, or KRAS Q61L mutation.

Optionally, the pharmaceutically acceptable salt is a tartrate salt.

Optionally, the medicament is used in combination with a KRAS inhibitor, and further in combination with an effective amount of the KRAS inhibitor.

Optionally, the KRAS inhibitor is BI 1701963, JNJ-74699157, MRTX1257, MRTX849 (CAS No. 2326521-71-3), AMG510, D1553 (CAS No. 2559761-14-5), MRTX1133 (CAS No. 2621928-55-8), RMC-4998 (CAS No. 2642037-07-6), Divarasib (CAS No. 2417987-45-0), LY3537982 (CAS No. 2414198-64-2), Opnurasib (CAS No. 2653994-08-0), or a pharmaceutically acceptable salt thereof, and the AMG510 has a structure of:

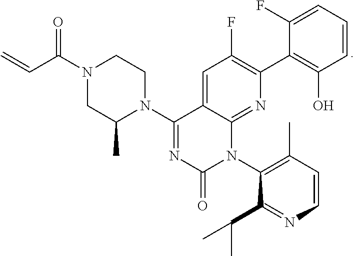

Optionally, the KRAS inhibitor is AMG510 or a pharmaceutically acceptable salt thereof. Optionally, the KRAS inhibitor is D1553 or a pharmaceutically acceptable salt thereof. Optionally, the KRAS inhibitor is MRTX1133 or a pharmaceutically acceptable salt thereof. Optionally, the KRAS inhibitor is RMC-4998 or a pharmaceutically acceptable salt thereof. Optionally, the KRAS inhibitor is Divarasib or a pharmaceutically acceptable salt thereof. Optionally, the KRAS inhibitor is LY3537982 or a pharmaceutically acceptable salt thereof. Optionally, the KRAS inhibitor is Opnurasib or a pharmaceutically acceptable salt thereof. Optionally, the KRAS inhibitor is MRTX849 or a pharmaceutically acceptable salt thereof.

Optionally, the tumor is lung cancer such as lung adenocarcinoma and non-small cell lung cancer, colorectal cancer, gastric cancer such as gastric adenocarcinoma cancer or pancreatic cancer with a KRAS mutation.

Optionally, the KRAS mutation is a G12C mutation. Optionally, the KRAS mutation is a G12D mutation.

Optionally, the medicament is used in combination with an MEK inhibitor, and further in combination with an effective amount of the MEK inhibitor.

Optionally, the MEK inhibitor is trametinib, cobimetinib, binimetinib, selumetinib, PD-325901, TAK-733, or a pharmaceutically acceptable salt thereof.

Optionally, the MEK inhibitor is trametinib or cobimetinib.

Optionally, the tumor is lung cancer, colorectal cancer or pancreatic cancer with a KRAS mutation.

Optionally, the KRAS mutation is a G12C mutation.

In another aspect, the present disclosure provides a method of treating a tumor such as a tumor with a KRAS mutation comprising administering to a subject an effective amount of a compound or a pharmaceutically acceptable salt thereof, wherein the compound has a structure of:

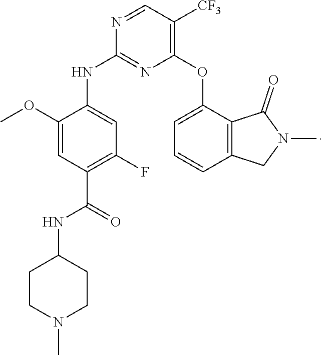

Optionally, the tumor is pancreatic cancer, colorectal cancer, lung cancer such as lung adenocarcinoma and non-small cell lung cancer, kidney cancer, gastric cancer such as gastric adenocarcinoma cancer, prostate cancer or ovarian cancer.

Optionally, the KRAS mutation is a G12A, G12C, G12D, G12R, G12S, G12V, G13C, G13D, G13V, Q61K, Q61L, Q61R or Q61H mutation.

Optionally, the KRAS mutation is a G12C, G12D, G13C or Q61K mutation. Optionally, the KRAS mutation is a G12C mutation. Optionally, the KRAS mutation is a G12D mutation.

Optionally, the tumor is 1) lung cancer, colorectal cancer or pancreatic cancer with a KRAS G12C mutation; 2) acute myeloid leukemia with a KRAS G12D, KRAS G12V, KRAS G13D or KRAS Q61H mutation; 3) bladder cancer with a KRAS G12C, KRAS G12D, KRAS G12R, KRAS G12V, KRAS G13D, or KRAS Q61H mutation; 4) breast cancer with a KRAS G12C, or KRAS G12V mutation; 5) cervical cancer with a KRAS G12C, KRAS G12D, KRAS G12V, or KRAS G13D mutation; 6) bile duct cancer with a KRAS G12R, or KRAS Q61K mutation; 7) colorectal cancer with a KRAS G12A, KRAS G12C, KRAS G12D, or KRAS G13D mutation; 8) esophageal cancer with a KRAS G12D mutation; 9) gastric cancer with a KRAS G12C, KRAS G12D, KRAS G12S, KRAS G12V, KRAS G13D, or KRAS Q61H mutation; 10) glioblastoma with a KRAS G12D mutation; 11) liver cancer with a KRAS G12C, KRAS G12D, or KRAS G13D mutation; 12) lung cancer with a KRAS G12A, KRAS G12D, KRAS G12S, KRAS G12V, KRAS G13C, KRAS G13D, KRAS Q61K, or KRAS Q61L mutation; 13) melanoma with a KRAS G12C, KRAS G12D, KRAS G12R, KRAS G13D, KRAS Q61K, KRAS Q61L, or KRAS Q61R mutation; 14) mesothelioma with a KRAS G12C mutation; 15) ovarian cancer with a KRAS G12R, KRAS G12V, KRAS Q61L, or KRAS G13C mutation; 16) pancreatic cancer with a KRAS G12A, KRAS G12D, KRAS G12R, KRAS G12V, KRAS G13C, or KRAS Q61H mutation; 17) prostate cancer with a KRAS G12D, KRAS G12R, or KRAS G12V mutation; 18) kidney cancer with a KRAS G12C, KRAS G12D, or KRAS G12V mutation; 19) sarcoma with a KRAS G13C, or KRAS Q61H mutation; 20) thyroid cancer with a KRAS G12V, KRAS Q61K, or KRAS Q61R mutation; 21) testicular cancer with a KRAS G12A, KRAS G12R, KRAS G12S, KRAS G12V, KRAS Q61L, or KRAS Q61R mutation; 22) thymoma with a KRAS G12D mutation; or 23) metrocarcinoma with a KRAS G12A, KRAS G12C, KRAS G12D, KRAS G12S, KRAS G12V, KRAS G13C, KRAS G13D, KRAS G13V, KRAS Q61H, or KRAS Q61L mutation.

Optionally, the method further comprises administering to the subject an effective amount of a KRAS inhibitor.

Optionally, the KRAS inhibitor is BI 1701963, JNJ-74699157, MRTX1257, MRTX849, AMG510, D1553, MRTX1133, RMC-4998, Divarasib, LY3537982, Opnurasib or a pharmaceutically acceptable salt thereof, and the structure of the AMG510 is as follows:

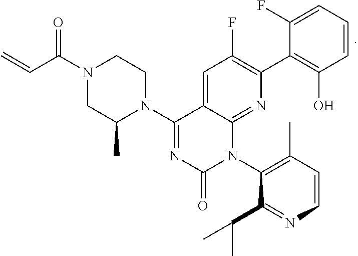

Optionally, the KRAS inhibitor is AMG510 or a pharmaceutically acceptable salt thereof. Optionally, the KRAS inhibitor is D1553 or a pharmaceutically acceptable salt thereof. Optionally, the KRAS inhibitor is MRTX1133 or a pharmaceutically acceptable salt thereof. Optionally, the KRAS inhibitor is RMC-4998 or a pharmaceutically acceptable salt thereof. Optionally, the KRAS inhibitor is Divarasib or a pharmaceutically acceptable salt thereof. Optionally, the KRAS inhibitor is LY3537982 or a pharmaceutically acceptable salt thereof. Optionally, the KRAS inhibitor is Opnurasib or a pharmaceutically acceptable salt thereof. Optionally, the KRAS inhibitor is MRTX849 or a pharmaceutically acceptable salt thereof.

Optionally, the tumor is lung cancer such as lung adenocarcinoma and non-small cell lung cancer, colorectal cancer, gastric cancer such as gastric adenocarcinoma cancer or pancreatic cancer with a KRAS mutation.

Optionally, the tumor is pancreatic cancer with a KRAS G12C mutation. Optionally, the tumor is pancreatic cancer with a KRAS G12D mutation.

Optionally, the tumor is colorectal cancer with a KRAS G12C mutation. Optionally, the tumor is colorectal cancer with a KRAS G12D mutation.

Optionally, the tumor is lung cancer, such as lung adenocarcinoma and non-small cell lung cancer, with a KRAS G12C mutation. Optionally, the tumor is lung cancer, such as lung adenocarcinoma and non-small cell lung cancer, with a KRAS G12D mutation.

Optionally, the tumor is gastric cancer, such as gastric adenocarcinoma cancer, with a KRAS G12C mutation. Optionally, the tumor is gastric cancer, such as gastric adenocarcinoma cancer, with a KRAS G12D mutation.

Optionally, the compound or a pharmaceutically acceptable salt thereof and the KRAS inhibitor are administered simultaneously, alternately or sequentially.

Optionally, the method further comprises administering to the subject an effective amount of an MEK inhibitor.

Optionally, the MEK inhibitor is trametinib, cobimetinib, binimetinib, selumetinib, PD-325901, TAK-733, or a pharmaceutically acceptable salt thereof.

Optionally, the MEK inhibitor is trametinib or cobimetinib.

Optionally, the tumor is lung cancer, colorectal cancer or pancreatic cancer with a KRAS mutation.

Optionally, the tumor is pancreatic cancer with a KRAS G12C mutation, colorectal cancer with a KRAS G12C mutation, or lung cancer with a KRAS Q61K mutation.

Optionally, the compound or a pharmaceutically acceptable salt thereof and the KRAS inhibitor are administered simultaneously, alternately or sequentially.

Optionally, the pharmaceutically acceptable salt is a tartrate salt.

In another aspect, the present disclosure provides a method for treating a tumor with a KRAS mutation, comprising steps of:
a) assessing whether a tissue sample obtained from the subject's cancer has a KRAS mutation; and
b) administering to the subject an effective amount of the compound or a pharmaceutically acceptable salt thereof if the cancer has a KRAS mutation;
wherein the compound has a structure of:

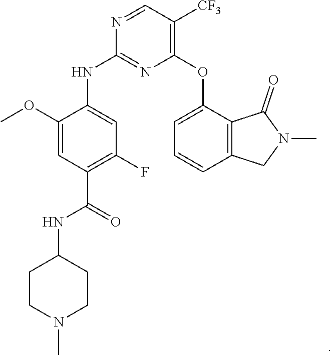

Optionally, the method further includes c) if the cancer is not characterized by a KRAS mutation, then an anticancer agent other than FAK inhibitors should be administered.

In another aspect, the present disclosure provides a pharmaceutical composition comprising a compound or a pharmaceutically acceptable salt thereof, and a KRAS inhibitor, wherein the compound has a structure of:

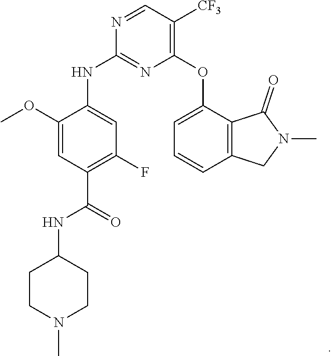

Optionally, the KRAS inhibitor is BI 1701963, MRTX849, AMG510, D1553, MRTX1133, RMC-4998, Divarasib, LY3537982, Opnurasib or a pharmaceutically acceptable salt thereof, and the AMG510 has a structure of:

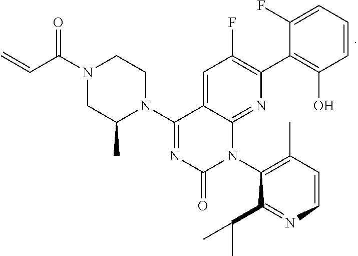

Optionally, the KRAS inhibitor is AMG510 or a pharmaceutically acceptable salt thereof. Optionally, the KRAS inhibitor is D1553 or a pharmaceutically acceptable salt thereof. Optionally, the KRAS inhibitor is MRTX1133 or a pharmaceutically acceptable salt thereof. Optionally, the KRAS inhibitor is RMC-4998 or a pharmaceutically acceptable salt thereof. Optionally, the KRAS inhibitor is Divarasib or a pharmaceutically acceptable salt thereof. Optionally, the KRAS inhibitor is LY3537982 or a pharmaceutically acceptable salt thereof. Optionally, the KRAS inhibitor is Opnurasib or a pharmaceutically acceptable salt thereof. Optionally, the KRAS inhibitor is MRTX849 or a pharmaceutically acceptable salt thereof.

In another aspect, the present disclosure provides a pharmaceutical composition comprising a compound or a pharmaceutically acceptable salt thereof, and an MEK inhibitor, wherein the compound has a structure of:

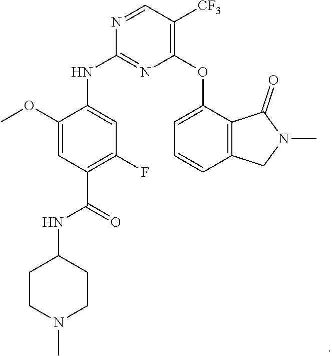

Optionally, the MEK inhibitor is trametinib, cobimetinib, binimetinib, selumetinib, PD-325901, TAK-733, or a pharmaceutically acceptable salt thereof.

Optionally, the MEK inhibitor is trametinib or a pharmaceutically acceptable salt thereof.

Optionally, the pharmaceutically acceptable salt is a tartrate salt.

In another aspect, the present disclosure provides a combination of pharmaceutical compositions comprising a first pharmaceutical composition comprising a compound or a pharmaceutically acceptable salt thereof, and a second pharmaceutical composition comprising a KRAS inhibitor or a pharmaceutically acceptable salt thereof, wherein the compound has a structure of:

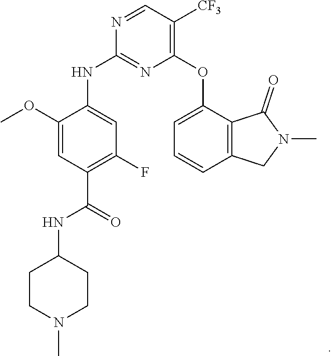

Optionally, the KRAS inhibitor in the second pharmaceutical composition is BI 1701963, MRTX849, AMG510, D1553, MRTX1133, RMC-4998, Divarasib, LY3537982, Opnurasib or a pharmaceutically acceptable salt thereof.

Optionally, the KRAS inhibitor in the second pharmaceutical composition is D1553 or a pharmaceutically acceptable salt thereof.

Optionally, the KRAS inhibitor in the second pharmaceutical composition is MRTX1133 or a pharmaceutically acceptable salt thereof.

Optionally, the KRAS inhibitor in the second pharmaceutical composition is MRTX849 or a pharmaceutically acceptable salt thereof.

Optionally, the pharmaceutically acceptable salt of the compound in the first pharmaceutical composition is a tartrate salt.

In another aspect, the present disclosure provides the use of a compound or a pharmaceutically acceptable salt thereof with a KRAS inhibitor or an MEK inhibitor in the manufacture of a medicament for treating a tumor such as a tumor with a KRAS mutation, wherein the compound has a structure of:

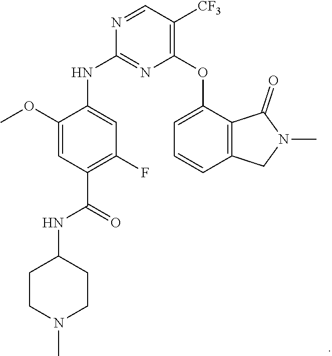

In another aspect, the present disclosure provides the use of a compound or a pharmaceutically acceptable salt thereof in the manufacture of a medicament in combination with a KRAS inhibitor or an MEK inhibitor for treating a tumor such as a tumor with a KRAS mutation, wherein the compound has a structure of:

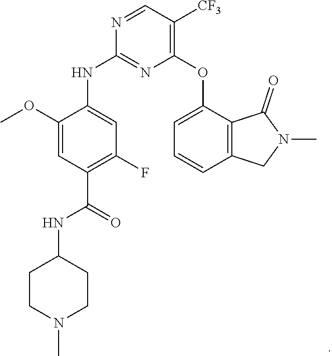

In another aspect, the present disclosure provides the use of a KRAS inhibitor or an MEK inhibitor in the manufacture of a medicament in combination with a compound or a pharmaceutically acceptable salt thereof for treating a tumor such as a tumor with a KRAS mutation, wherein the compound has a structure of:

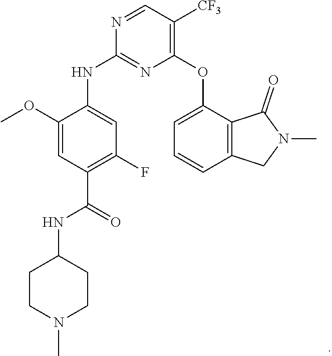

In another aspect, the present disclosure provides the following compound or a KRAS inhibitor or an MEK inhibitor or a combination thereof for use in the treatment of a tumor such as a tumor with a KRAS mutation, wherein the compound has a structure of:

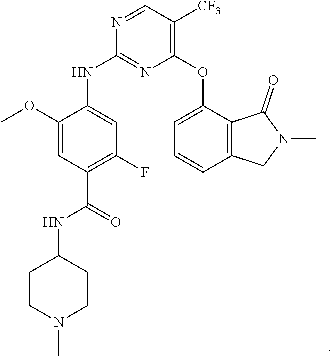

Optionally, the KRAS inhibitor or MEK inhibitor is as defined herein, and the mutation is as defined herein, and the tumor is as defined herein.

### BRIEF DESCRIPTION OF THE DRAWINGS

FIG. 1 depicts the anti-tumor effects of the compound, which illustrates that the compound showed tumor growth inhibition (TGI, %) at a dose of 50 mg/kg once a day in nude mouse xenograft models of human pancreatic cancer cells MIA PaCa-2 (G12C mutation), lung cancer cells Calu-6 (Q61K mutation) and HCC-461 (G12D mutation), and ovarian cancer cells TOV-21G (G13C mutation).

FIG. 2 depicts the tumor growth kinetics of the nude mouse xenograft model of pancreatic cancer cell MIA PaCa-2, which illustrates the antitumor effects of different test groups in the nude mouse xenograft model of pancreatic cancer cell MIA PaCa-2 (Example 2). The test groups were the control group; the compound group, administered once a day with a dose of 25 mg/kg; the AMG510 group, administered once a day with a dose of 10 mg/kg; the combined administration group of the compound and AMG510, with the compound administered once a day at a dose of 25 mg/kg and AMG510 administered once a day at a dose of 10 mg/kg; respectively.

FIG. 3 depicts the tumor volume (mm³) of animals in the nude mouse xenograft model of pancreatic cancer cell MIA PaCa-2 at day 11, which illustrates the antitumor effects of different test groups in the nude mouse xenograft model of pancreatic cancer cell MIA PaCa-2 (Example 2). The test groups were the control group; the compound group, administered once a day with a dose of 25 mg/kg; the AMG510 group, administered once a day with a dose of 10 mg/kg; the combined administration group of the compound and AMG510, with the compound administered once a day at a dose of 25 mg/kg and AMG510 administered once a day at a dose of 10 mg/kg; respectively.

FIG. 4 depicts the mean tumor volume (mm³) of the nude mouse xenograft model of lung cancer cell Calu-6, which illustrates the antitumor effect of different test groups in the nude mouse xenograft model of lung cancer cell Calu-6 (Example 3). The test groups were the control group; the compound group, administered once a day with a dose of 50 mg/kg; the trametinib group, administered once a day with a dose of 0.125 mg/kg; the combined administration group of the compound and trametinib, with the compound administered once a day at a dose of 50 mg/kg and trametinib administered once a day at a dose of 0.125 mg/kg; respectively.

FIG. 5 depicts the tumor growth kinetics of the nude mouse patient derived xenograft model of colorectal cancer CO-04-0070, which illustrates the antitumor effects of different test groups in the nude mouse xenograft model of colorectal cancer CO-04-0070 (Example 4). The test groups were the control group; the compound group, administered once a day with a dose of 25 mg/kg; the AMG510 group, administered once a day with a dose of 30 mg/kg; the combined administration group of the compound and AMG510, with the compound administered once a day at a dose of 25 mg/kg and AMG510 administered once a day at a dose of 30 mg/kg; respectively.

FIG. 6 depicts percent change in body weights (%) of different treatment groups in female BALB/c nude mice bearing PC-07-0013 established tumors. The changes were calculated based on the animal weights of the first day of dosing (PG-D0). Data points represent percent group mean change in body weight. Error bars represent standard error of the mean (SEM).

FIG. 7 depicts the tumor growth curves of different treatment groups of female BALB/c nude mice bearing PC-07-0013 established tumors. Data points represent group mean, error bars represent standard error of the mean (SEM).

FIG. 8 depicts tumor growth curve after dosing of CO-04-0070 subcutaneous xenograft tumor model in female BALB/c nude mouse. Data points represent the average tumor volume within the group, and error bars represent standard error (SEM). All animals in groups 1 and 2 were euthanized and tumor tissues were collected on day 19 after dosing; the #91563 animal in group 3 was euthanized and tumor tissues were collected on day 55 after dosing due to weight loss exceeded 20%; and the remaining animals in group 3 and all animals in group 4 were euthanized and tumor tissues were collected on day 70 after dosing.

FIG. 9 depicts tumor growth curve of AGS tumor-bearing mice post administration of test articles. Data were expressed as mean±SEM.

FIG. 10 depicts tumor weight changes of the tumor-bearing mice in different groups. Data were expressed as mean±SEM.

FIG. 11 depicts the effects of various concentrations of KRAS G12C inhibitors in combination with the compound on cell killing in NCIH-2122.

FIG. 12 depicts the effects of various concentrations of KRAS G12C inhibitors in combination with the compound on cell killing in NCI-H358.

FIG. 13 depicts the tumor growth curve of NCI-H2122 tumor-bearing mice in different groups. Data were expressed as mean±SEM.

FIG. 14 depicts the tumor weight of the tumor-bearing mice in different groups. Data were expressed as mean±SEM.

FIG. 15 depicts the tumor growth curve of MIA PaCa-2 tumor-bearing mice in different groups. Data were expressed as mean±SEM.

FIG. 16 depicts the tumor weight of the tumor-bearing mice in different groups. Data were expressed as mean±SEM.

FIG. 17 depicts the percentage change of the body weights of different treatment groups in female BALB/c Nude mice bearing CO-04-0070 established tumors. The changes were calculated based on the animal weights of the first day of dosing (PG-D0). Data points represent percent group mean change in body weight. Error bars represent standard error of the mean (SEM).

FIG. 18 depicts the tumor growth curves of G1 to G4 groups of female BALB/c Nude mice bearing CO-04-0070 established tumors. Data points represent group mean, error bars represent standard error of the mean (SEM).

### DETAILED DESCRIPTION OF THE INVENTION

### Embodiments and Definitions

The present disclosure relates to the use of a compound or a pharmaceutically acceptable salt thereof in the manufacture of a medicament for treating a tumor such as a tumor with a KRAS mutation. The present disclosure also relates to a method of treating a tumor such as a tumor with a KRAS mutation comprising administering to a subject an effective amount of a compound or a pharmaceutically acceptable salt thereof as defined herein.

The term “KRAS” as used herein refers to the Kirsten rat sarcoma viral oncogene homolog, a protein belonging to the RAS gene family that encodes a small G protein having intrinsic GTPase activity and contributes to the activation of downstream effectors involving multiple pathways, including apoptosis, proliferation, and differentiation.

As used herein, the term “KRAS mutation” refers to a mutation in KRAS, i.e., one or more changes in the amino acid sequence of wild-type KRAS. Mutations in KRAS can lead to loss of intrinsic GTPase activity and thus to dysregulation of cellular proliferative signaling. In some embodiments, KRAS is mutated at one or more positions selected from codons 12, 13, 59, and 61. In some embodiments, KRAS is mutated at one or more amino acid positions selected from G12, G13, S17, P34, A59, and Q61. In some embodiments, KRAS is mutated at one or more amino acid positions selected from G12C, G12S, G12R, G12F, G12L, G12N, G12A, G12D, G12V, G13C, G13S, G13D, G13V, G13P, S17G, P34S, A59E, A59G, A59T, Q61K, Q61L, Q61R, and Q61H. In some embodiments, KRAS is mutated at one or more amino acid positions selected from G12, G13, A59, Q61, K117, and A146. In some embodiments, KRAS is mutated at one or more amino acid positions selected from G12C, G12R, G12S, G12A, G12D, G12V, G13C, G13R, G13S, G13A, G13D, G13V, A59E, A59G, A59T, Q61K, Q61L, Q61R, Q61H, K117N, K117R, K117E, A146P, A146T, and A146V. In some embodiments, KRAS is mutated at one or more amino acid positions selected from G12, G13, A59, and Q61. In some embodiments, KRAS is mutated at one or more amino acid positions selected from G12C, G12R, G12S, G12A, G12D, G12V, G13C, G13R, G13S, G13A, G13D, A59E, A59G, A59T, Q61K, Q61L, Q61R, and Q61H. In some embodiments, KRAS is mutated at one or more amino acid positions selected from G12, G13, and D153. In some embodiments, KRAS is mutated at one or more amino acid positions selected from G12A, G12C, G12D, G12V, G12S, G13D, and D153V. In some embodiments, KRAS is mutated at one or more amino acid positions selected from G12C, G12S, and D153V.

In some embodiments, the KRAS mutation occurs in G12A, G12C, G12D, G12R, G12S, G12V, G13C, G13D, G13V, Q61K, Q61L, Q61R, or Q61H. In some embodiments, the KRAS mutation occurs in G12C, G12D, G13C, orQ61K.

Whether a cancer has a KRAS mutation is determined by conventional diagnostic methods for obtaining the cancer cells from a patient, including but not limited to biopsies, blood tests, and other diagnostic methods. Through these methods, samples of cancer cells, such as tissue samples, circulating tumor cells or biomolecules with characteristics of cancer (eg circulating nucleic acids) are obtained, and whether KRAS mutations are present in the cancer cells is then determined.

Methods of characterizing KRAS mutations, e.g., detection, sequencing, analysis of the KRAS gene or its expression (e.g., DNA, mRNA), include KRAS nucleic acid amplification and/or visualization. To detect the KRAS gene or its expression, nucleic acid can be isolated from a subject by conventional methods in the art, and the isolated nucleic acid can then be amplified (e.g., by polymerase chain reaction (PCR) (such as direct PCR, quantitative real-time PCR and reverse transcriptase PCR), ligase chain reaction, self-sustaining sequence replication, transcription amplification systems, Q-Beta replicase, etc.) and visualized (e.g., by labeling nucleic acids during amplification, exposure to intercalating compounds/dyes, probes). Another method for detecting KRAS mutations in codons 12 and 13 is the commercially available THERASCREEN™, a KRAS mutation test kit (DxS Limited, Manchester, UK). Additional methods for detecting KRAS mutations are disclosed in Detmer et al, US20180223377 and Huang et al., US20180036304, the entire teachings of which are incorporated herein by reference.

Another embodiment of the present disclosure is the use of the compound or a pharmaceutically acceptable salt thereof in combination with an MEK inhibitor.

The term “MEK” as used herein refers to a mitogen-activated protein kinase, a serine-threonine kinase, which mediates intracellular signaling involved in the regulation of protein and cellular functions associated with membranes, intracellular and intercellular process as well as transformation, proliferation/growth, differentiation, survival and death.

The term “MEK inhibitor” as used herein refers to a compound or agent that reduces MEK-dependent cell signaling/function, and reduces MEK-associated tumor cell proliferation/growth. Examples of MEK inhibitors include, but are not limited to, trametinib, cobimetinib, binimetinib, selumetinib, PD-325901, TAK-733, or a pharmaceutically acceptable salt thereof.

The term “KRAS inhibitor” as used herein refers to a chemical or agent that reduces KRAS activity (e.g., GTPase activity) and results in a decrease in KRAS-related apoptosis, proliferation and differentiation. Examples of KRAS inhibitors include but are not limited to BI 1701963, JNJ-74699157, MRTX1257, MRTX849, AMG510, D1553, MRTX1133, RMC-4998, Divarasib, LY3537982, Opnurasib or a pharmaceutically acceptable salt thereof. In some embodiments, examples of KRAS inhibitors include but are not limited to BI1701963, MRTX849, AMG510 or a pharmaceutically acceptable salt thereof. The BI1701963 was developed by Boehringer Ingelheim, Germany, with the US Clinical Trial Database (ClinicalTrials.gov) register number of NCT04111458. The MRTX849 was developed by the US biotechnology company Mirati, with the US Clinical Trial Database register number of NCT03785249. The AMG510 has a structure of:

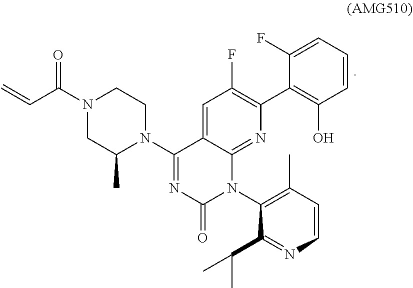

The compound, or a pharmaceutically acceptable salt thereof, and the MEK inhibitor (or KRAS inhibitor) can be administered simultaneously, alternately, or sequentially.

The compound, or a pharmaceutically acceptable salts thereof, can be combined with other antiproliferative agents or anticancer therapies to treat tumors. The antiproliferative or anticancer therapy includes surgery, radiation therapy (including but not limited to gamma radiation, neutron beam radiation therapy, electron beam radiation therapy, proton therapy, brachytherapy and systemic radioisotopes), endocrine therapy, biological response modifiers (including but not limited to interferons, interleukins, and tumor necrosis factors (TNF)), hyperthermia and cryotherapy, agents to reduce any adverse effects (e.g., antiemetics), and other approved chemotherapy agents.

The compound and a pharmaceutically acceptable salts thereof can be used alone or in combination with an MEK inhibitor or a KRAS inhibitor to treat KRAS-mutant tumors. The term “KRAS-mutant tumor” as used herein refers to a tumor with a KRAS mutation. KRAS mutations result in loss of intrinsic GTPase activity and/or dysregulation of cellular proliferative signaling. In some embodiments, the tumor is selected from the group consisting of colorectal cancer, pancreatic cancer, kidney cancer, lung cancer, liver cancer, breast cancer, prostate cancer, gastrointestinal cancer, peritoneal cancer, melanoma, endometrial cancer, ovarian cancer, cervical cancer, uterine cancer, bladder cancer, glioblastoma, brain metastases, salivary gland cancer, thyroid cancer, brain cancer, lymphoma, myeloma and head and neck cancer. In some embodiments, the tumor is selected from squamous cell carcinoma, small cell lung cancer, non-small cell lung cancer, lung adenocarcinoma, lung squamous carcinoma, hepatocellular carcinoma, colon cancer, endometrial cancer, and hepatocellular carcinoma. In some embodiments, the tumor is selected from the group consisting of colorectal cancer, prostate cancer, breast cancer, lung cancer, endometrial cancer, multiple myeloma, pancreatic cancer, renal cancer, and glioblastoma. In some embodiments, the tumor is selected from non-small cell lung cancer, pancreatic cancer, and glioblastoma. In some embodiments, the tumor is non-small cell lung cancer. In some embodiments, the KRAS-mutant tumor is pancreatic cancer, colorectal cancer, lung cancer, kidney cancer, gastric cancer, prostate cancer, ovarian cancer, breast cancer, brain cancer, bladder cancer, cervical cancer, esophageal cancer, liver cancer, thyroid cancer , testicular cancer, uterine cancer, thymoma, hepatocellular carcinoma, head and neck cancer, bile duct cancer, neuroblastoma, melanoma, glioblastoma, lymphoma, leukemia, acute myeloid leukemia, melanoma, mesothelioma, sarcoma, paraganglioma, osteosarcoma, germ cell tumor, or mesothelioma. In some embodiments, the KRAS-mutant tumor is pancreatic cancer, colorectal cancer, lung cancer, kidney cancer, gastric cancer, prostate cancer, or ovarian cancer.

In some embodiments, specific examples of the KRAS-mutant tumor include 1) lung cancer, colorectal cancer or pancreatic cancer with a KRAS G12C mutation; 2) acute myeloid leukemia with a KRAS G12D, KRAS G12V, KRAS G13D or KRAS Q61H mutation; 3) bladder cancer with a KRAS G12C, KRAS G12D, KRAS G12R, KRAS G12V, KRAS G13D, or KRAS Q61H mutation; 4) breast cancer with a KRAS G12C, or KRAS G12V mutation; 5) cervical cancer with a KRAS G12C, KRAS G12D, KRAS G12V, or KRAS G13D mutation; 6) bile duct cancer with a KRAS G12R, or KRAS Q61K mutation; 7) colorectal cancer with a KRAS G12A, KRAS G12C, KRAS G12D, or KRAS G13D mutation; 8) esophageal cancer with a KRAS G12D mutation; 9) gastric cancer with a KRAS G12C, KRAS G12D, KRAS G12S, KRAS G12V, KRAS G13D, or KRAS Q61H mutation; 10) glioblastoma with a KRAS G12D mutation; 11) liver cancer with a KRAS G12C, KRAS G12D, or KRAS G13D mutation; 12) lung cancer with a KRAS G12A, KRAS G12D, KRAS G12S, KRAS G12V, KRAS G13C, KRAS G13D, KRAS Q61K, or KRAS Q61L mutation; 13) melanoma with a KRAS G12C, KRAS G12D, KRAS G12R, KRAS G13D, KRAS Q61K, KRAS Q61L, or KRAS Q61R mutation; 14) mesothelioma with a KRAS G12C mutation; 15) ovarian cancer with a KRAS G12R, KRAS G12V, KRAS Q61L, or KRAS G13C mutation; 16) pancreatic cancer with a KRAS G12A, KRAS G12D, KRAS G12R, KRAS G12V, KRAS G13C, or KRAS Q61H mutation; 17) prostate cancer with a KRAS G12D, KRAS G12R, or KRAS G12V mutation; 18) kidney cancer with a KRAS G12C, KRAS G12D, or KRAS G12V mutation; 19) sarcoma with a KRAS G13C, or KRAS Q61H mutation; 20) thyroid cancer with a KRAS G12V, KRAS Q61K, or KRAS Q61R mutation; 21) testicular cancer with a KRAS G12A, KRAS G12R, KRAS G12S, KRAS G12V, KRAS Q61L, or KRAS Q61R mutation; 22) thymoma with a KRAS G12D mutation; or 23) metrocarcinoma with a KRAS G12A, KRAS G12C, KRAS G12D, KRAS G12S, KRAS G12V, KRAS G13C, KRAS G13D, KRAS G13V, KRAS Q61H, or KRAS Q61L mutation.

In some embodiments, the compound or a pharmaceutically acceptable salt thereof of the present disclosure is used in combination with an MEK inhibitor (e.g., trametinib, cobimetinib, binimetinib, selumetinib, PD-325901, TAK-733, especially trametinib) for the treatment of KRAS-mutant lung, colorectal or pancreatic cancer. The KRAS mutation can be any one of the above-mentioned mutations. In one embodiment, the KRAS mutation is a G12C mutation.

In some embodiments, the compound, or a pharmaceutically acceptable salt thereof, is used in combination with a KRAS inhibitor (e.g., BI 1701963, MRTX849, D1553, MRTX1133, RMC-4998, Divarasib, LY3537982, Opnurasib, and AMG510, especially AMG510) for the treatment of KRAS-mutant lung, gastric, colorectal, or pancreatic cancer. The KRAS mutation can be any one of the above-mentioned mutations. In one embodiment, the KRAS mutation is a G12C mutation.

As used herein, the term “pharmaceutically acceptable” means non-toxic, biologically tolerable and suitable for administration to a subject.

As used herein, the term “pharmaceutically acceptable salt” refers to a salt that is non-toxic, biologically tolerable and suitable for administration to a subject. The pharmaceutically acceptable salts of the compounds refer to an acid addition salt that is non-toxic, biologically tolerable and suitable for administration to a subject, including but not limited to: acid addition salts formed by the compounds with an inorganic acid, such as hydrochloride, hydrobromide, carbonate, bicarbonate, phosphate, sulfate, sulfite, nitrate, and the like, as well as acid addition salts formed by the compounds with an organic acid, such as formate, acetate, malate, maleate, fumarate, tartrate, succinate, citrate, lactate, methanesulfonate, p-toluenesulfonate, 2-hydroxyethanesulfonate, benzoate, salicylate, stearate, and salts formed with alkane-dicarboxylic acid of formula HOOC—(CH₂)ₙ—COOH (wherein n is 0-4), etc. Pharmaceutically acceptable salts can be obtained by conventional methods well known in the art, for example by reacting a sufficiently basic compound such as an amine with a suitable acid which provides a physiologically acceptable anion. In some embodiments, the salt is a tartrate salt.

As used herein, the term “subject” refers to mammals and non-mammals. Mammals means any member of the mammalian class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex. In some embodiments, the subject is a human.

The term “treat”, “treating” or “treatment” as used herein refers to obtaining a desired pharmacological and/or physiological effect. The effect may be therapeutic and includes partial or substantial achievement of one or more of the following: partial or total reduction in the extent of the disease, condition or syndrome; improvement in clinical symptoms or indicators associated with the disease; or delaying, inhibiting or reducing likelihood of progression of the disease, condition or syndrome.

The term “effective amount” as used herein refers to an amount of the compound (or a pharmaceutically acceptable salt thereof), an MEK inhibitor and/or a KRAS inhibitor sufficient to reduce or ameliorate the severity, duration, progression, or onset of the disease or condition, to delay or arrest the progression of the disease or condition, to cause regression of the disease or condition or delay the recurrence or progression of symptoms, or to enhance or improve the therapeutic effect of another therapy. The precise amount of the compound (or a pharmaceutically acceptable salt thereof), MEK inhibitor and/or KRAS inhibitor administered to a subject will depend on various factors, such as the given agent or compound, pharmaceutic preparation, route of administration, the type of disease, the condition, the identity of the subject or host being treated, etc., but can still be routinely determined by those skilled in the art. For example, determination of an effective amount will also depend on the degree, severity, and type of cell proliferation. The skilled artisan will be able to determine the appropriate dosage based on these and other factors. When co-administered with other therapeutic agents, e.g., when co-administered with an anticancer agent, the “effective amount” of any other therapeutic agent will depend on the type of the agent used. Appropriate dosages are known for approved therapeutics and can be adjusted by the skilled artisan depending on the condition of the subject, the type of condition being treated, and the amount of the compound or a pharmaceutically acceptable salt thereof. In cases where the amount is not explicitly stated, the amount should be assumed to be an effective amount. An effective dose of the compound or a pharmaceutically acceptable salt thereof may range from 10 μg to 2000 mg. This example is non-limiting. Effective amounts of MEK inhibitors and KRAS inhibitors are known to those skilled in the art.

The compounds or pharmaceutically acceptable salts thereof can be administered by any suitable method of administration. Suitable methods include oral, enteral, parenteral, intravenous, intramuscular or subcutaneous administration to the subject.

Thus, the compound or a pharmaceutically acceptable salt thereof can be administered orally with a pharmaceutically acceptable carrier such as an inert diluent or an absorbable edible carrier. They can be enclosed in hard-or soft-shell gelatin capsules, compressed into tablets, or mixed directly with the patient's food. For oral therapeutic administration, the compound, or a pharmaceutically acceptable salt thereof, can be combined with one or more excipients and used in a form of ingestible tablets, buccal tablets, lozenges, capsules, elixirs, suspensions, syrups or wafers. These preparations contain an effective amount of the compound.

Tablets, lozenges, pills, capsules, etc. may further comprise: binders such as tragacanth, acacia, cornstarch or gelatin; excipients such as dicalcium phosphate; disintegrants such as corn starch, potato starch, alginic acid, etc .; lubricants, such as magnesium stearate; or sweeteners, such as sucrose, fructose, lactose or aspartame; or flavoring agents.

The compounds may also be administered intravenously or intraperitoneally by infusion or injection. Solutions of the compounds can be prepared in water, optionally mixed with nontoxic surfactants.

Exemplary pharmaceutical dosage forms for injection or infusion include: sterile aqueous solutions, dispersions, or sterile powders containing the active ingredient suitable for the extemporaneous preparation of sterile injectable or infusion solutions or dispersions. In any event, the final dosage form should be sterile, fluid, and stable under the conditions of manufacture and storage.

Sterile injectable solutions can be prepared by incorporating a required amount of the compound in an appropriate solvent together with other desired ingredients enumerated above and then being filtrated and sterilized. In the case of sterile powders for preparing sterile injectable solutions, the preferred methods of preparation may be vacuum drying and the freeze-drying techniques, which yield a powder of the active ingredient plus any other desired ingredients previously present after sterile filtration.

The amount of compound required for treatment may vary not only with the particular salt chosen, but also with the route of administration, the nature of the disease being treated, and the age and condition of the patient, and is ultimately at the discretion of the attending physician or clinician. However, the dosage may generally range from about 0.1 to about 50 mg/kg body weight per day.

The desired dose may conveniently be presented as a single dose or as divided doses for administration at appropriate intervals.

The term “FAK inhibitor” as used herein refers to a potent inhibitor of FAK, which may be suitable for mammals, particularly humans.

### EXAMPLES

The following examples are provided to further illustrate the present disclosure. It should be understood that these examples are only used to illustrate the present disclosure and not to limit the scope of the present disclosure.

The experimental methods without specific conditions in the following examples can be carried out according to the conventional conditions of this type of reaction or according to the conditions suggested by the manufacturer.

The experimental materials and reagents used in the following examples can be obtained from commercial sources unless otherwise specified.

The meanings of the abbreviations used in the examples are as follows:
ATCC American Type Culture Collection
CO₂ Carbon dioxide
d Days
FCS Fetal Calf Serum
kg Kilograms
l Liter
mg Milligram
ml Milliliter
mm³ Cubic millimeter
PBS Phosphate Buffered Saline
RPMI 1640 RPMI 1640 Medium
p.o. & PO Oral
qd/QD Daily
TGI Tumor Growth Inhibition
i.v. Intravenous injection
i.p. & IP Intraperitoneal injection
s.c. Subcutaneous injection
Bid Twice a day
BIW Twice a week
QW Once a week
TGI_{TV} Relative tumor inhibition rate
SPF Specific pathogen free
mm millimeter
μL microliter
° C. Degree Centigrade
RT Room temperature
FBS Fetal Bovine Serum
mpk mg/kg
n Number
N/A Not applicable
P.S. Penicillin-Streptomycin
SEM Standard error of mean
T/C Relative tumor growth rate
IC₅₀ median inhibition concentration

### Test Articles

| | | | | |
|---|---|---|---|---|
| Name | BI853520 | MRTX1133 | D1553 | RMC-4998 |
| Mark | The compound | MRTX1133 | | RMC-4998 |
| Vendor | Prepared | Shanghai | Prepared according | MCE |
| | according to | SuperLan | to WO2020233592 | |
| | WO2010058032 | Chemical Co., | | |
| | | Ltd. | | |
| Cat/Lot | | Lot: 0179-cgm- | | Cat: HY-156671 |
| | | 51-B | | Lot: 317630 |

| | | | | |
|---|---|---|---|---|
| Name | Divarasib | LY3537982 | Opnurasib | AMG510 |
| Mark | Divarasib | LY3537982 | Opnurasib | AMG510 |
| Vendor | MCE | MCE | MCE | Selleck |
| Cat/Lot | Cat: HY-145928 | Cat: HY-132980 | Cat: HY-139612 | Lot: S883001 |
| | Lot: 265847 | Lot: 263272 | Lot: 263272 | |

| | |
|---|---|
| Name | MRTX849 |
| Mark | MRTX849 |
| Vendor | Nantong HI- |
| | FUTURE Biology |
| | Co., Ltd |
| Cat/Lot | Lot: HF2023101500 |

### Generic Xenograft Models for Antitumor Study

The study used a nude mouse xenograft model of human cancer cells with KRAS mutations.

Athymic Female Bom Tac: approximately 6-week-old NMRI-Foxnlnu mice were purchased from Taconic, Denmark. After arriving in the animal room, mice were acclimated to the new environment for at least 3 days before being used for assay. The animals were housed under standard conditions (temperature 21.5±1.5° C. and 55±10% humidity) with 5 mice in each group. The animals were provided standard diet and autoclaved tap water ad libitum. A Datamars T-IS 8010 FDX-B transponder implanted subcutaneously in the neck region and a LabMax II fixed reader were used to identify each mouse. The cage card showing the study number, animal identification number, compound and dose level, route of administration, and dosing schedule for the animal throughout the assay was retained on the animals.

To establish subcutaneous tumors, Human cancer cells having a KRAS mutation were harvested by trypsinization, centrifuged, washed and suspended in ice-cold PBS +5% FCS and growth factor reduced Matrigel (1:1) at a cell concentration of 1×10⁸ cells/ml. Then 100 μl of cell suspension containing 2.5×10⁶−1×10⁷ cells was injected subcutaneously into the right flank of nude mice (1 site per mouse). When tumors were established and reached a diameter of 6-8 mm (7 days after cell injection), mice were randomly assigned to the treatment group and the control group.

Compounds suspended in 1M HCl and diluted with 0.5% hydroxyethyl cellulose were administered intragastrically through gavage needle every day, and the dosage was 10 mL/Kg.

Tumor diameters were measured with calipers three times a week (Monday, Wednesday, and Friday). The volume of each tumor [in mm³] was calculated according to the equation, “tumor volume=length×diameter²×π/6”. To monitor the side effects of the treatment, the mice were checked daily for abnormalities and their body weights were measured three times a week (Monday, Wednesday, and Friday). Animals were sacrificed at the end of the study (approximately three weeks after the start of treatment). During the study animals with tumor necrosis or tumors larger than 2000 mm³ were sacrificed ahead of schedule for ethical reasons.

At the end of the assay, statistical evaluation of tumor volume and body weight parameters was performed. Absolute tumor volume and percent change in body weight (referenced to initial weight on day 1) were used. A nonparametric approach was used, and the number of observations, median, minimum and maximum values were calculated. For a quick overview of possible treatment effects, the median tumor volume for each treatment group T and the median tumor volume for the control group C were used to calculate the TGI from day 1 to day d:
Relative tumor volume:(T/C)

$$T / C = 100 \times \frac{{T}_{d}}{{C}_{d}}$$

TGI from day 1 to day d:

$$TGI = 100 \times \frac{( {C}_{d} - {C}_{1} ) - ( {T}_{d} - {T}_{1} )}{( {C}_{d} - {C}_{1} )}$$

wherein,
C₁, T₁=median tumor volume in control and treatment groups at the start of the assay (day 1).

C_{d}, T_{d}=median tumor volume in control and treatment groups at the end of the assay (day d).

Each dose of test compound was compared to the control group using a one-sided descending wilcoxon test, taking reduction in tumor volume as a treatment effect and weight loss as a side effect. The P-values for tumor volume (the efficacy parameter) were compared and adjusted for multiple times according to Bonferroni-Holm, while the P-values for body weight (the tolerance parameter) were not adjusted so as not to overlook possible side effects. Significance level was fixed at α=5%. A p-value (adjusted) of less than 0.05 was considered to show a statistically significant difference between groups, and 0.05≤p-value<0.10 was considered as an indicative difference. Statistical evaluations were performed using the software packages SAS version 9.2 (SAS Institute Inc., Cary, NC, USA) and Proc StatXact version 8.0 (Cytel Software, Cambridge, MA, USA).

### Example 1: Antitumor Activity Study of the Compound in Xenograft Models

This study followed the procedure described in Generic Xenograft Models for Antitumor Study.

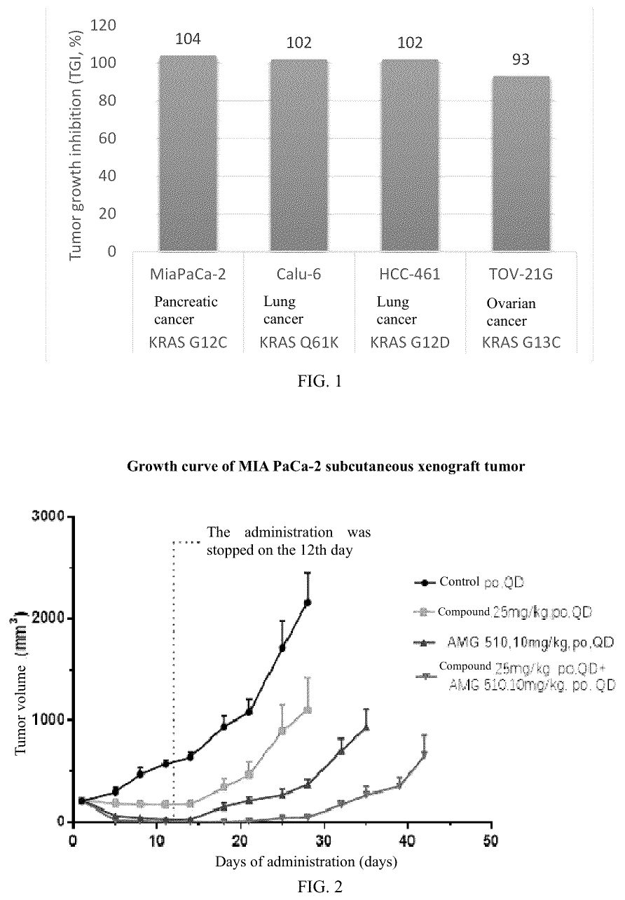

The cell lines used in this assay were obtained from ATCC, including pancreatic cancer cells MIA PaCa-2 (G12C mutation), lung cancer cells Calu-6 (Q61K mutation) and HCC-461 (G12D mutation), and ovarian cancer cells TOV-21G (G13C mutation) (FIG. 1). A master cell bank and a working cell bank (WCB) were established according to BI RCV GmbH & Co KG standards. Cells were grown in T175 tissue culture flasks using the following media:
MIA PaCa-2: DMEM+GlutaMax, supplemented with 10% heat-inactivated fetal bovine serum, 1% non-essential amino acids and 1% sodium pyruvate;
TOV-21G: MCDB 105 medium containing 1.5 g/L sodium bicarbonate was mixed with medium 199 containing 15% heat-inactivated FCS at a ratio of 1:1;
Calu-6: MEM+1% NEAA+1% sodium pyruvate+1% GlutaMax+10% FCS;
HCC-461: RPMI-1640/GlutaMAX, 10% FCS.

Cells were cultured in humidified air at 37° C. and 5% CO₂. Cultures were maintained at a concentration of 8×10⁶ cells/ml to 12×10⁷ cells/ml. The number of inoculations of each cell to establish a tumor model was as follows: MIA PaCa-2 (1×10⁷), TOV-21G (5×10⁶), Calu-6 (2.5×10⁶), and HCC-461 (5×10⁶).

Five independent tests were performed. In each test, ten or twelve tumor-bearing mice in each group were treated with either the control formulation or the compound, respectively. Treatment was initiated when the median tumor volume reached approximately 50-100 mm³ and terminated after 2 to 4 weeks. The compound in the treatment group was suspended in 0.5% hydroxyethyl cellulose and administered daily at a dose of 50 mg/kg, while the control group was treated with 0.5% hydroxyethyl cellulose alone.

After treatment with 50 mg/kg of the compound once a day, the TGI of pancreatic cancer cells MIA PaCa-2, lung cancer cells Calu-6 and HCC-461, and ovarian cancer cells TOV-21G ranged from 93% to 104% (p <0.0001) (FIG. 1).

### Example 2: Antitumor Study of the Compound and KRAS Inhibitors in the MIA PaCa-2 Cell Line Xenograft Model

This study followed the procedure described in Generic Xenograft Models for Antitumor Study.

Human pancreatic cancer MIA PaCa-2 cells harboring the KRAS G12C mutation were obtained from ATCC. A master cell bank (MCB) and a working cell bank (WCB) were established according to BI RCV GmbH & Co KG standards. Cells used in each assay were obtained from WCB 16.10.2006 (Lab Tontsch-Grunt) or WCB 11.02.2009 (Lab Hirt). Cells were grown in T175 tissue culture flasks containing DMEM+GlutaMax supplemented with 10% heat-inactivated fetal bovine serum, 1% non-essential amino acids and 1% sodium pyruvate. Cells were cultured in humidified air at 37° C. and 5% CO₂. Cultures were maintained at a concentration of 1×10⁶ cells/ml to 3×10⁶ cells/ml.

Each group of 4 tumor-bearing mice was treated as follows: the control group (treated with 0.5% hydroxyethyl cellulose), the compound group (suspended in 0.5% hydroxyethyl cellulose, administered daily at a dose of 25 mg/kg), the AMG510 (KRAS inhibitor) group (suspended in 0.5% hydroxyethyl cellulose, administered daily at a dose of 10 mg/kg), and the combination group of the compound (suspended in 0.5% hydroxyethyl cellulose, administered daily at a dose of 25 mg/kg) and AMG510 (suspended in 0.5% hydroxyethyl cellulose, administered daily at a dose of 10 mg/kg). The treatment was initiated when the median volume of the tumor reached about 200 mm³. After 11 days of continuous administration, the administration was stopped on the 12th day, and the tumor growth of the mice was continued to be observed.

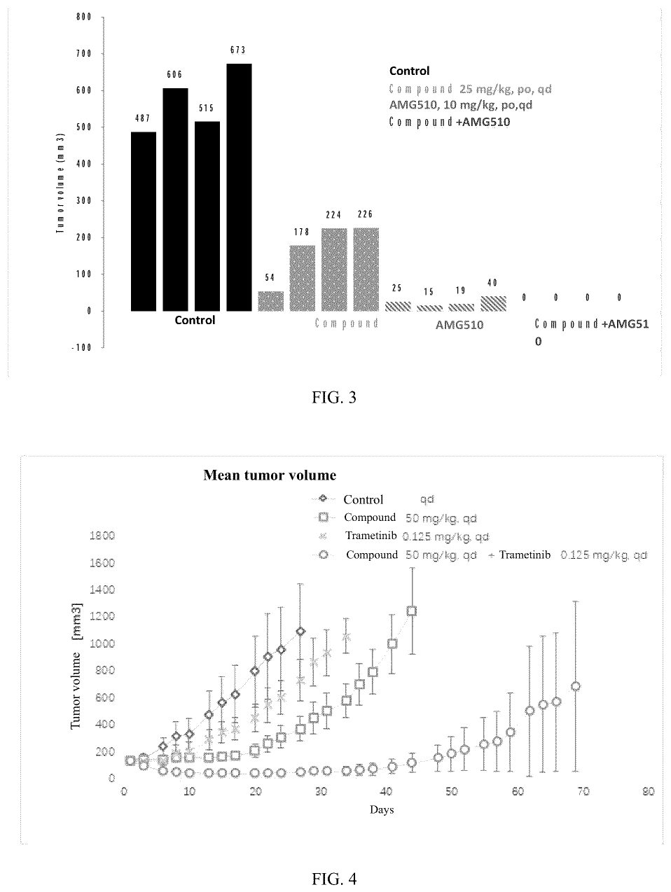

100% of tumors disappeared in the combination group of the compound at 25 mg/kg and AMG510 at 10 mg/kg (FIG. 2 and FIG. 3).

In the MIA PaCa-2 cell model of pancreatic cancer with a KRAS G12C mutation, combination of the compound and the KRAS inhibitor showed good synergistic effect.

### Example 3: Antitumor Study of the Compound and MEK Inhibitors in the Calu-6 Cell Line Xenograft Model

This study followed the procedure described in Generic Xenograft Models for Antitumor Study.

Human lung cancer Calu-6 cells harboring the KRAS Q61K mutation were obtained from ATCC. A master cell bank (MCB) and a working cell bank (WCB) were established according to BI RCV GmbH & Co KG standards. Cells used in each assay were obtained from WCB 16.10.2006 (Lab Tontsch-Grunt) or WCB 11.02.2009 (Lab Hirt). Cells were grown in T175 tissue culture flasks containing MEM+1% NEAA+1% sodium pyruvate +1% GlutaMax+10% FCS as medium. Cells were cultured in humidified air at 37° C. and 5% CO2. Cultures were maintained at a concentration of 2.5×10⁶ cells/ml.

Each group of 10 tumor-bearing mice was treated as follows: the control group (treated with 0.5% hydroxyethyl cellulose), the compound group (suspended in 0.5% hydroxyethyl cellulose, administered daily at a dose of 50 mg/kg), the trametinib (MEK inhibitor) group (suspended in 0.5% hydroxyethyl cellulose, administered daily at a dose of 0.125 mg/kg), and the combination group of the compound (suspended in 0.5% hydroxyethyl cellulose, administered daily at 50 mg/kg) and AMG510 (suspended in 0.5% hydroxyethyl cellulose, administered daily at a dose of 0.125 mg/kg). Treatment was initiated when the median tumor volume reached approximately 200 mm³ and terminated when the tumor volume exceeded 1000 mm³.

Tumor volume was significantly reduced in the combination group of the compound at 50 mg/kg and trametinib at 0.125 mg/kg (FIG. 4).

In the Calu-6 cell model of lung cancer with a KRAS Q61K mutation, combination of the compound and the MEK inhibitor showed good synergistic effect.

### Example 4: Antitumor Study of the Compound and KRAS Inhibitors in the CO-04-0070 (KRAS G12C Mutation) Subcutaneous Xenograft Model

6-to 8-week-old BALB/c nude mice were purchased from Beijing Vital River Laboratory Animal Technology Co., Ltd. After arriving in the animal room, mice were acclimated to the new environment for at least 3 days before being used for the assay. The animals were housed under standard conditions (temperature 20-26° C. and 40-70% humidity) with 5 animals per group. The animal information card for each cage indicated the quantity, sex, strain, date of receipt, dosing schedule, experiment number, group, and the start date of the assay for the animals in the cage. All cages, litter and drinking water were sterilized prior to use. Cages, feed and drinking water were changed twice a week. Animals in the assay were identified by ear tags.

The human-derived colorectal cancer CO-04-0070 model was originally derived from tumor samples resected during clinical surgery. After the surgically resected tumor specimens were sequenced for mutation and gene expression using next-generation sequencing technology, the tumor tissue was cut into 20-30 mm³, and then inoculated into several mice. Tumor tissue that can grow smoothly would be excised and cryopreserved in liquid nitrogen for subsequent resuscitation.

The nomenclature of passage was that nude mice inoculated with tumor samples was P0 generation, which continued to passage as P1 generation, and so on, and the resuscitated specimen was named as FP. The tumor tissue used in this assay was the FP3 generation. A 20-30 mm³ of CO-04-0070 FP3 tumor tissue mass was subcutaneously inoculated into the right back of each mouse, and tumor growth was awaited. Randomization was performed for groups when the mean tumor volume reached about 112 mm³ (Table 1).

TABLE 1
Animal grouping and dosing schedule

| | | | | Dosing | | |
|---|---|---|---|---|---|---|
| | | | | volume | | |
| | | Compound | Dosage | parameters | Route of | Dosing |
| Group | N¹ | therapy | (mg/kg) | (μL/g)² | administration | frequency |
| 1 | 4 | Control | — | 10 | PO | QD × 22 |
| 2 | 4 | The compound | 25 | 10 | PO | QD × 22 |
| 3 | 5 | AMG510 | 30 | 10 | PO | QD × 33 |
| 4 | 5 | The compound + | 25 + 30 | 10 | PO | QD × 33 |
| | | AMG510 | | | | |

Note:
¹N: The quantity of mice in each group;
²Dosing volume: 10 μL/g based on mouse body weight.

Tumor-bearing mice in each group were treated as follows: the control group (treated with 0.5% hydroxyethyl cellulose), the AMG510 (KRAS inhibitor) group (suspended in 0.5% hydroxyethyl cellulose, administered daily at a dose of 30 mg/kg), and the combination group of the compound (suspended in 0.5% hydroxyethyl cellulose, administered daily at a dose of 25 mg/kg) and AMG510 (suspended in 0.5% hydroxyethyl cellulose, administered daily at a dose of 30 mg/kg).

The indicator of the assay was to examine whether tumor growth was inhibited, delayed or cured. Tumor diameters were measured with vernier calipers twice a week. The equation for calculating tumor volume was: V=0.5a×b², wherein a and b represented the long and short diameters of the tumor, respectively.

The antitumor efficacy of the compounds was evaluated by TGI (%) or tumor proliferation rate T/C (%).

TGI(%), reflecting tumor growth inhibition rate, was calculated:

TGI(%)=[1−(mean tumor volume at the end of administration in a certain treatment group−mean tumor volume at the beginning of administration in this treatment group)/(mean tumor volume at the end of treatment in the control group−mean tumor volume at the beginning of treatment in the control group)]×100%.

Tumor proliferation rate T/C (%) was calculated as follows:

$$T / C ( \% ) = Ti / Vi \times 100$$

Wherein, Vi is the mean tumor volume of the solvent control group in a certain measurement, and Ti is the mean tumor volume of the administration group in the same measurement.

After the assay, the tumor weights were detected, and the percentages of T/C_{weight} were calculated. T_{weight} and C_{weight} represented the tumor weight of the administration group and the solvent control group, respectively.

Statistical analysis includes mean and standard error of mean (SEM) of tumor volume at each time point for each group. Statistical analysis was performed to evaluate the differences between groups based on the data on the 21st day after the start of administration, and one-way ANOVA was used to analyze the comparison between multiple groups. Because of the significant difference in F value, the Games-Howell method was used for test. All data analyses were performed with SPSS 17.0, and p<0.05 was considered as a significant difference.

### Results of Assay

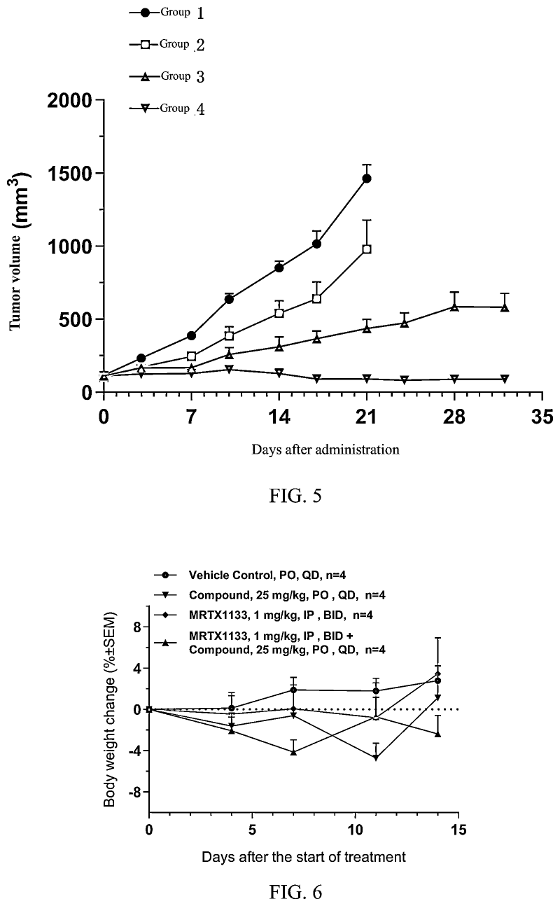

On the 21st day after the start of administration, the tumor volume of the tumor-bearing mice in the control group reached 1,388 mm³. Compared with the control group, the tumor volume of animals in group 2, group 3 and group 4 was 978 mm³ (T/C=66.89%, TGI=35.34%, p=0.04), 436 mm³ (T/C=31.38%, TGI=74.63%, p=0.001), and 91 mm³ (T/C=6.57%, TGI=101.65%, p=0.001), respectively, all of which had tumor-suppressive effects (FIG. 5).

In the CO-04-0070 model of colorectal cancer with a KRAS G12C mutation, combination of the compound and the KRAS inhibitor AMG510 showed good synergistic effect.

### Example 5: Evaluation of Anti-Tumor Efficacy of the Compound (“IN10018”) and MRTX1133 in the PC-07-0013 Human Pancreatic Cancer Patient-Derived Xenograft (PDX) Model in Female BALB/c Nude Mice

The objective of the research was to evaluate the in vivo anti-tumor efficacy of the compound and MRTX1133 in the subcutaneous human pancreatic cancer PDX model of PC-07-0013 in female BALB/c nude mice.

### Group and Regimen

TABLE 2
Group and regimen

| | | | | Dosage | | |
|---|---|---|---|---|---|---|
| | | | Dosage | Volume | | |
| Group | N¹ | Test Article | (mg/kg) | (mL/kg)² | Route | Schedule |
| 1 | 4 | Vehicle | N/A | 10 | PO | BID × 14 |
| | | | | | | Days |
| 2 | 4 | The compound | 25 | 10 | PO | QD*14 Days |
| 3 | 4 | MTRX1133 | 1 | 10 | IP | BID * 29 |
| | | | | | | Doses |
| 4 | 4 | MRTX1133 + | 1 + 25 | 10 | IP + | BID * 29 |
| | | The compound | | | PO | Doses + |
| | | | | | | QD * 14 |
| | | | | | | Days |

Note:
¹N: Number of mice per group;
²Dosage Volume: Based on the body weight 10 ml/kg, the administration will be suspended when the mice body weight change rate decreases to 15% and will be continued to dose until the body weight change rate recovers to reduce by 10%.

6-to 8-week-old female BALB/c nude mice were purchased from Zhejiang Vital River Laboratory Animal Technology Co., Ltd. After the animals arrived, they were isolated in the experimental environment for at least 3 days before starting the experiment. The mice were kept in Individual Ventilation Cages (4 animals in each cage) at constant temperature (20-26° C.) and humidity (40-70%) with a 12 hours light and 12 hours dark cycle. Animals had free access to sterile food and water during the entire study period. Animals were marked by ear tags.

### Generation of the PDX Model

The pancreatic cancer PDX model of PC-07-0013 was originally established from a surgically resected clinical sample, and implanted in NOD SCID mice defined as passage 0 (PO). The next passage implanted from P0 tumor was defined as passage 1 (P1), and so on during continual implantation in mice. The FP5 tumor tissue was used for this study.

### Tumor Inoculation and Animal Grouping

Each mouse was implanted subcutaneously at the right flank with the PC-07-0013 FP5 tumor slices (˜30 mm³) for tumor development. Treatment was started on day 21 after tumor implantation when the average tumor size reached approximately 110 mm³. The animals were assigned into groups using an Excel-based randomization software performing stratified randomization based upon their tumor volumes. Each group consisted of 4 tumor-bearing mice. The test articles were administrated to the mice according to the predetermined regimens as shown in the experimental design table. (Table 2)

### Observation

At the time of routine monitoring, the animals were daily checked for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset.

### Tumor Measurements and Endpoints

The major endpoint was to see if the tumor growth could be delayed or mice could be cured. Tumor size was measured twice weekly in two dimensions using a caliper, and the volume was expressed in mm3 using the formula: V=0.5 a×b² where a and b are the long and short diameters of the tumor, respectively. The tumor sizes were then used for the calculations of both TGI (%) and the T/C (%) values.

TGI was calculated for each group using the formula: TGI (%)=[1−(Ti−T0)/(Vi−V0)]×100, wherein Ti is the average tumor volume of a treatment group on a given day, T0 is the average tumor volume of the treatment group on the day of treatment starts, Vi is the average tumor volume of the control group on the same day with Ti, and V0 is the average tumor volume of the control group on the day of treatment starts.

The T/C value (in percent) is an indication of antitumor effectiveness, wherein T and C are the mean volumes of the treated and control groups, respectively, on a given day.

### Statistical Analysis

Data of tumor volume and body weight were expressed as Mean±SEM. All data was analyzed with GraphPad Prism and the statistical analysis of differences of tumor volume in each group during the whole experiment was conducted by Two-way ANOVA, and Fisher's LSD test. P<0.05 was considered to be statistically significant.

### Results

### Mortality, Morbidity, and Body Weight Gain or Loss

Animal body weight was monitored regularly as an indicator of toxicity. There were no deaths and no morbidity. All mice did not show obvious body weight loss. The body weight changes of different treatment groups were shown in FIG. 6.

### Tumor Volume

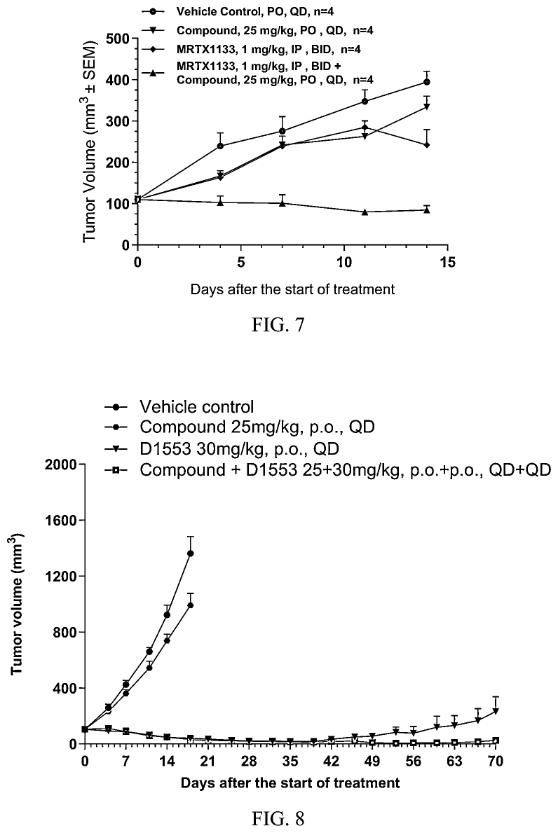

Mean tumor volumes over time in female BALB/c nude mice bearing PC-070013 xenografts tumor dosed with the compound and MRTX1133 were shown in Table 3, Table 4 and FIG. 7.

TABLE 3
Tumor volumes over time
Tumor size (mm³)¹

| | | | | MRTX1133 + |
|---|---|---|---|---|
| | | The compound | MRTX1133 | The compound |
| Days | Vehicle | 25 mg/kg | 1/0.5 mg/kg | 1 ± 25 mg/kg |
| 0 | 110 ± 8 | 110 ± 8 | 110 ± 10 | 110 ± 6 |
| 4 | 239 ± 32 | 167 ± 13 | 163 ± 16 | 103 ± 16 |
| 7 | 276 ± 35 | 242 ± 6 | 239 ± 25 | 101 ± 20 |
| 11 | 348 ± 28 | 263 ± 19 | 284 ± 16 | 80 ± 7 |
| 14 | 394 ± 26 | 334 ± 26 | 242 ± 37 | 84 ± 11 |

Note:
¹Mean ± SEM.

TABLE 4
Tumor growth inhibition calculation for the compound and
MRTX1133 in the PC-07-0013 PDX model
calculated based on tumor volume measurements at day 14

| | Tumor | | | | |
|---|---|---|---|---|---|
| | Size | | | | |
| | (mm³)¹ | T/C² | TGI³ | | |
| Treatment | at day 14 | (%) | (%) | p value⁴ | p value⁵ |
| Vehicle | 394 ± 26 | — | — | — | — |
| The compound, | 334 ± 26 | 84.77 | 21.18 | 0.0437 * | <0.0001 **** |
| 25 mg/kg | | | | | |
| MRTX1133, | 242 ± 37 | 61.42 | 53.58 | <0.0001 **** | <0.0001 **** |
| 1 mg/kg | | | | | |
| MRTX1133, | 84 ± 11 | 21.32 | 108.98 | <0.0001 **** | — |
| 1 mg/kg + | | | | | |
| The compound, | | | | | |
| 25 mg/kg | | | | | |

Note:
¹Mean ± SEM;
²Tumor Growth Inhibition was calculated by dividing the group average tumor
volume for the treated group by the group average tumor volume for the control group (T/C);
³TGI (%) = [1 − (T₁₄ − T₀)/(V₁₄ − V₀)] × 100%;
⁴* p < 0.05, **** p < 0.0001, Two-way ANOVA, vs. Vehicle group;
⁵**** p < 0.0001, Two-way ANOVA, vs. MRTX1133, 1 mg/kg + The compound, 25 mg/kg group.

### Conclusion

The 2-drug combination therapy showed the best anti-tumor efficacy among all the tested groups. The efficacy data indicated that the compound synergizes with MRTX1133 in the treatment of cancer.

The compound and MRTX1133 at dose levels were tolerated well by the tumor-bearing mice in this study. No obvious body weight loss was observed in all treatment groups.

### Example 6: In Vivo Efficacy Study of the Compound in Human-Derived Colorectal Cancer CO-04-0070 Subcutaneous Xenograft Tumor Model in BALB/C Nude Mice

The aim of this study was to evaluate the in vivo efficacy of the compound in human-derived colorectal cancer CO-04-0070 subcutaneous xenograft tumor model in BALB/C nude mice.

### Grouping and Dosing Schedule

TABLE 5
Animal grouping and dosing schedule

| | | | | Dosing | | |
|---|---|---|---|---|---|---|
| | | | | volume | Route of | |
| | | Test | Dosage | parameters | adminis- | Dosing |
| Group | N¹ | Article | (mg/kg) | (μL/g)² | tration | frequency |
| 1 | 5 | Vehicle¹ | — | 10 | PO | QD × 20 |
| 2 | 5 | The | 25 | 10 | PO | QD × 20 |
| | | compound | | | | |
| 3 | 5 | D1553 | 30 | 10 | PO | QD × 71 |
| 4 | 5 | The | 25 + 30 | 10 | PO | QD × 71 |
| | | compound + | | | | |
| | | D1553 | | | | |

Note:
¹N: The quantity of mice in each group;
²Dosing volume: 10 μL/g based on mouse body weight. The administration will be suspended when the mice body weight change rate decreases to 15% and will be continued to dose until the body weight change rate recovers to reduce by 10%.

Tumor collection: after the experiment, all animals were euthanized and tumor tissues were collected, photographed and weighed. All tumor blocks were divided into two parts, and used for FFPE preparation and protein extraction, respectively. The samples of groups 1 and 2 were collected on day 19 after the start of dosing; the #91563 animal in group 3 was euthanized and samples were collected on day 55 after the start of dosing due to weight loss exceeded 20%; and the remaining animals in group 3 and all animals in group 4 were euthanized and samples were collected on day 70 after the start of dosing.

6-to 8-week-old female BALB/c nude mice, weighted 17-23 g, were purchased from Beijing Vital River Laboratory Animal Technology Co., Ltd. After the animals arrived, they were isolated in the experimental environment for 3 days before starting the experiment. The mice were kept in Individual Ventilation Cages (5 animals in each cage) at constant temperature (20-26° C.) and humidity (40%-70%) with a 12 hours light and 12 hours dark cycle. Animals had free access to irradiation sterilized dry granule food and sterile drinking water during the entire study period. The identification labels for each cage contained the following information: number of animals, sex, strain, date received, treatment, study number, group number and the starting date of the treatment. Animals were marked by ear tags.

### Establishment of PDX Model

The human-derived colorectal cancer CO-04-0070 model was originally derived from tumor samples resected during clinical surgery. The passage naming rule is as follows: the tumor sample inoculated in nude mice is named passage 0 (PO), and the next passage is named P1, and so on. The resuscitated specimen is named FP. The tumor tissue used in this experiment was FP6.

### Tumor Inoculation

20-30 mm³ CO-04-0070 FP6 tumor tissue block was subcutaneously inoculated on the right back of each mouse for tumor growth. When the average tumor volume reached about 105 mm³, random grouping and dosing was started (Table 2.5).

### Observations

At the time of routine monitoring, the animals were daily checked for any effects of tumor growth and treatments on normal behavior such as mobility, food and water consumption (by looking only), appearance and any other abnormal effects as stated in the protocol. Death and side effects were recorded on the basis of the numbers of animals within each subset.

### Tumor Measurements and the Endpoints

The major endpoint is to see if the tumor growth can be inhibited or delayed, or if mice can be cured. Tumor sizes were measured twice weekly in two dimensions using a caliper, and the volume was expressed in mm³ using the formula: V=0.5×a×b² (a, long diameters; b, short diameters).

The tumor sizes were then used for the calculations of TGI (%) or T/C (%) values as antitumor effect of the compound. TGI (%) represents inhibition rate of tumor growth, which was calculated using the formula: TGI (%)=[1−(average tumor volume of a treatment group at the end of dosing−average tumor volume of the treatment group at the start of dosing)/(average tumor volume of the control group at the end of dosing−average tumor volume of the control group at the start of dosing)]×100%.

T/C (%) was calculated using the formula: T/C (%)=Tᵢ/Vᵢ×100, wherein Vᵢ is the average tumor volume of the control group on a given day, and Tᵢ is the average tumor volume of a treatment group on the same day.

At the end of the experiment, the tumor weight was measured, and the percentage of T_{weight}/C_{weight} was calculated, wherein T_{weight} and C_{weight} represent the tumor weight of the treatment group and the control group, respectively.

### Statistical Analysis

Summary statistics, including mean and the standard error of the mean (SEM), are provided for the tumor volume of each group at each time point. The differences between groups were evaluated by statistical analysis based on the data on day 18 after the start of dosing, and the comparison between multiple groups was analyzed with one-way ANOVA. Due to the significant difference in F value, the Games-Howell method was used. All data were analyzed using SPSS 17.0, and P<0.05 was considered statistically significant.

### Results

The change of tumor volume, antitumor efficacy evaluation, and tumor weight in each group is shown in Table 6, Table 7, and Table 8. The tumor growth curve is shown in FIG. 8.

TABLE 6
Tumor volume at different time points in each group

| Days | | The | | The compound, |
|---|---|---|---|---|
| after | | compound | D1553, | 25 mg/kg + D1553, |
| dosing | Vehicle | 25 mg/kg | 30 mg/kg | 30 mg/kg |
| 0 | 104 ± 11 | 105 ± 19 | 104 ± 19 | 105 ± 20 |
| 4 | 258 ± 26 | 232 ± 27 | 93 ± 26 | 111 ± 19 |
| 7 | 425 ± 29 | 362 ± 25 | 89 ± 25 | 87 ± 12 |
| 11 | 659 ± 29 | 543 ± 49 | 65 ± 18 | 58 ± 8 |
| 14 | 923 ± 70 | 737 ± 48 | 46 ± 13 | 49 ± 8 |
| 18 | 1,361 ± 122 | 990 ± 86 | 40 ± 12 | 30 ± 5 |
| 21^{b} | — | — | 35 ± 9 | 24 ± 4 |
| 25 | — | — | 26 ± 7 | 23 ± 5 |
| 28 | — | — | 20 ± 5 | 16 ± 2 |
| 32 | — | — | 21 ± 3 | 16 ± 2 |
| 35 | — | — | 17 ± 3 | 14 ± 3 |
| 39 | — | — | 17 ± 5 | 12 ± 2 |
| 42 | — | — | 33 ± 8 | 18 ± 3 |
| 46 | — | — | 50 ± 12 | 19 ± 10 |
| 49 | — | — | 55 ± 18 | 10 ± 3 |
| 53 | — | — | 83 ± 39 | 4 ± 2 |
| 56 | — | — | 77 ± 48^{c} | 9 ± 3 |
| 60 | — | — | 119 ± 79 | 9 ± 4 |
| 63 | — | — | 132 ± 70 | 9 ± 3 |
| 67 | — | — | 167 ± 85 | 15 ± 4 |
| 70 | — | — | 232 ± 105 | 25 ± 7 |

Note:
^{a}Mean ± SEM;
^{b}All animals in groups 1 and 2 were euthanized and tumor tissues were collected on day 19 after dosing;
^{c}The #91563 animal in group 3 was euthanized and tumor tissues were collected on day 55 after dosing due to weight loss exceeded 20%.

TABLE 7
Antitumor efficacy evaluation of the compound and D1553 in human-
derived colorectal cancer CO-04-0070 xenograft tumor model
(calculated based on the tumor volume on 18^{th} day after dosing)

| | Tumor | | | |
|---|---|---|---|---|
| | volume | | | |
| | (mm³)^{a} | T/C^{b} | TGI^{c} | |
| Group | (at day 18) | (%) | (%) | P Value^{d} |
| Vehicle | 1,361 ± 122 | — | — | — |
| The compound, 25 mg/kg | 990 ± 86 | 72.76 | 29.57 | 0.243 |
| D1553, 30 mg/kg | 40 ± 12 | 2.95 | 105.08 | 0.002 |
| The compound, 25 mg/kg + | 30 ± 5 | 2.22 | 105.96 | 0.002 |
| D1553, 30 mg/kg | | | | |

Note:
^{a}Mean ± SEM;
^{b}Tumor growth inhibition was calculated by T/C;
^{c}TGI (%) = [1 − (T₁₈ − T₀)/(V₁₈ − V₀)] × 100);
^{d}p value was calculated based on tumor volume.

TABLE 8
Tumor weight in each group

| | Tumor weight (g)^{a} |
|---|---|
| Group | (at day 19^{b} and 70) |
| Vehicle | 1.520 ± 0.174 |
| The compound, 25 mg/kg | 1.153 ± 0.107 |
| D1553, 30 mg/kg | 0.229 ± 0.097^{c} |
| The compound, 25 mg/kg + D1553, 30 mg/kg | 0.019 ± 0.004 |

Note:
^{a}Mean ± SEM;
^{b}All animals in groups 1 and 2 were euthanized and tumor tissues were collected on day 19 after dosing;
^{c}The #91563 animal in group 3 was euthanized and tumor tissues were collected on day 55 after dosing due to weight loss exceeded 20%.

### Conclusion

On day 18 after dosing, the tumor volume of tumor-bearing mice in the control group reached 1,361 mm³. Compared with the control group, the tumor volumes of animals in group 3 (D1553, 30 mg/kg, PO, QD) and group 4 (the compound, 25 mg/kg+D1553, 30 mg/kg, PO, QD) were 40 mm³ (T/C=2.95%, TGI=105.08%, p=0.002) and 30 mm³ (T/C=2.22%, TGI=105.96%, p=0.002), respectively, which showed significant antitumor effect. The average tumor volume of group 2 (the compound, 25 mg/kg, PO, QD) was 990 mm³ (T/C=72.76%, TGI=29.57%, p=0.243), which did not show significant antitumor effect.

### Example 7: In Vivo Efficacy Study of MRTX1133 and the Compound in AGS Mouse Model

The aim of this study was to evaluate the anti-tumor efficacy of MRTX1133 and the compound in AGS model in NOG mice.

### Grouping and Dosing Schedule

TABLE 9
Detailed Information of Grouping and Treatments

| | | | Dose | Dose | | |
|---|---|---|---|---|---|---|
| | | | level | volume^{b} | Dose | Dose |
| Group | Test Article | N^{a} | (mg/kg) | (mL/kg) | Route | schedule |
| 1 | Vehicle (PBS) | 5 | N/A | 10 | p.o. | QD |
| 2 | MRTX1133 | 5 | 1 | 10 | i.p. | BID |
| 3 | The compound | 5 | 12.5 | 10 | p.o. | QD |
| 4 | MRTX1133 + | 5 | 1 + 12.5 | 10 + 10 | i.p. + | BID + QD |
| | The compound | | | | p.o | |

Note:
^{a}Number of animals per group;
^{b}Dosing volume: 10 mL/kg based on body weight. Animals administration were stopped when body weight loss exceeded 15%, and resumed when body weight returned within 10%.

6- to 8-week-old female Mus musculus NOG mice, weighted 18-21 g, were purchased from Beijing Vital River Laboratory Animal Technology Co., Ltd. The mice were kept in Individual Ventilation Cages (5 animals in each cage) at constant temperature (20-26° C.) and humidity (40%-70%). Animals had free access to irradiation sterilized dry granule food and sterile drinking water during the entire study period. The identification labels for each cage contained the following information: number of animals, sex, strain, date received, treatment, study number, group number and the starting date of the treatment. Animals were marked by ear tags.

### Cell Culture

AGS (human gastric adenocarcinoma cell line, from Medsyin cell bank) cells were maintained in vitro in RPMI-1640 medium supplemented with 10% FBS and 1% P.S. at 37° C. in an atmosphere of 5% CO₂ in air. The tumor cells were routinely sub-cultured twice or three times weekly. AGS cells growing in an exponential growth phase were resuspended in PBS and Matrigel (the volume ratio of PBS and Matrigel is 1:1) and adjusted concentration to 50×10⁶/mL for tumor injection.

### Tumor Inoculation and Administration

Each mouse was injected subcutaneously at the right flank with 0.2 mL AGS cell suspension (10×10⁶) for tumor development. When tumor reach around 190 mm³ on the 23^{rd} day after cell injection, the animals were randomly grouped according to tumor volume, followed by drug administration. The day of grouping was defined as day 0. Compounds administration and the animal numbers in each group were shown in Table 9.

### Observations

At the time of routine monitoring, the animals were daily checked for any effects of tumor growth and treatments on normal behavior such as mobility, food and water consumption (by looking only), eye/hair matting and any other abnormal effects as stated in the protocol. Death and observed clinical signs were recorded on the basis of the numbers of animals within each subset.

### Tumor Measurements and the Endpoints

The major endpoint is to see if the tumor growth can be delayed or mice can be cured. Tumor sizes were measured three times weekly in two dimensions using a caliper (No. 105688, sylvac Dantsin), and the volume was expressed in mm³ using the formula: V=0.5×a×b² (a, long diameters; b, short diameters). The tumor sizes were then used for the calculations of T/C (%) values. T/C (%) was calculated using the formula: T/C %=(Tᵢ/T₀)/(Vᵢ/V₀)×100% (%), wherein Tᵢ was the average tumor volume of a treatment group on a given day, T₀ was the average tumor volume of the treatment group on the first day of treatment, Vᵢ was the average tumor volume of the control group on the same day with Tᵢ, and V₀ was the average tumor volume of the control group on the first day of treatment. TGI was calculated for each group using the formula: TGI (%)=100%−(Tᵢ−T₀)/(Vᵢ−V₀)×100% (%).

### Sample Collection

The study was terminated on day 37 after treatment according to requirement, tumors from each mouse were collected and photographed, tumor weight were recorded meanwhile.

### Statistical Analysis

Summary statistics, including mean and the standard error of the mean (SEM), are provided for the tumor volume of each group at each time point. Statistical analysis of difference in tumor volume and tumor weight versus Vehicle group were conducted on the data obtained at each time point. T-test was performed to compare tumor volume among groups. A value of p<0.05 was considered to be statistically significant.

### Results

The study was terminated on day 37 after treatment. Tumor volume during these studies were shown as follows.

### Tumor Volumes

Tumor growth curves of AGS tumor-bearing mice after drug administration were shown in FIG. 9. On day 36 of experiment, the tumor volumes of the control group, MRTX1133 group, The compound group and MRTX1133+ The compound group were 1661.33 mm³, 619.45 mm³, 968.93 mm³ and 241.59 mm³, respectively.

Relative tumor volume (T/C, %) and tumor growth inhibition (TGI, %) were calculated at each time point and the values were shown in Table 10 and Table 11. Compared with the control group, the T/C value of MRTX1133 group, The compound group and MRTX1133+ The compound group on day 36 was 37.22%, 58.17 %and 14.53%, respectively. Correspondingly, the value of TGI was 70.86%, 47.11% and 96.54%, respectively. Statistical significance of each treatment group comparing with the control group was also analyzed and p values were shown in Table 12. Compared with Vehicle group, all treatment groups showed significant anti-tumor efficacy (p<0.05) after 36 days of treatment.

TABLE 10
T/C (%) after treatment

| T/C (%) Days after treatment | 0 | 2 | 5 | 8 | 11 | 15 | 18 | 22 | 25 | 29 | 32 | 36 |
|---|---|---|---|---|---|---|---|---|---|---|---|---|
| G2: MRTX1133, 1 mpk | — | 65.27 | 59.69 | 51.52 | 47.12 | 37.92 | 30.34 | 30.15 | 29.13 | 29.84 | 32.51 | 37.22 |
| G3: The compound, 12.5 mpk | — | 95.31 | 87.46 | 94.80 | 69.20 | 69.24 | 73.28 | 68.17 | 62.81 | 64.35 | 57.33 | 58.17 |
| G4: MRTX1133 + The | — | 46.72 | 44.08 | 39.13 | 31.96 | 20.14 | 16.35 | 14.04 | 12.65 | 14.65 | 13.63 | 14.53 |
| compound, 1 mpk + 12.5 mpk | | | | | | | | | | | | |

TABLE 11
TGI (%) after treatment

| TGI (%) Days after treatment | 0 | 2 | 5 | 8 | 11 | 15 | 18 | 22 | 25 | 29 | 32 | 36 |
|---|---|---|---|---|---|---|---|---|---|---|---|---|
| G2: MRTX1133, 1 mpk | — | 242.44 | 151.80 | 143.40 | 134.09 | 119.43 | 110.04 | 100.46 | 93.14 | 86.30 | 78.99 | 70.86 |
| G3: The compound, 12.5 mpk | — | 32.55 | 47.05 | 15.16 | 78.01 | 59.05 | 42.05 | 45.64 | 48.75 | 43.73 | 49.84 | 47.11 |
| G4: MRTX1133 + The | — | 371.72 | 210.52 | 180.00 | 172.51 | 153.61 | 132.13 | 123.64 | 114.83 | 105.01 | 101.14 | 96.54 |
| compound, 1 mpk + 12.5 mpk | | | | | | | | | | | | |

TABLE 12
P value in different groups at specific time point

| P value Days after treatment | 0 | 2 | 5 | 8 | 11 | 15 | 18 | 22 | 25 | 29 | 32 | 36 |
|---|---|---|---|---|---|---|---|---|---|---|---|---|
| G2: MRTX1133, 1 mpk | 0.9651 | 0.0092 | 0.0067 | 0.0081 | 0.0068 | 0.0057 | 0.0020 | 0.0015 | 0.0006 | 0.0001 | 0.0001 | 0.0000 |
| G3: The compound, 12.5 mpk | 0.9547 | 0.5641 | 0.3278 | 0.7364 | 0.1378 | 0.1823 | 0.2435 | 0.1421 | 0.0551 | 0.0320 | 0.0070 | 0.0039 |
| G4: MRTX1133 + The | 0.9918 | 0.0000 | 0.0002 | 0.0012 | 0.0010 | 0.0009 | 0.0005 | 0.0003 | 0.0001 | 0.0000 | 0.0000 | 0.0000 |
| compound, 1 mpk + 12.5 mpk | | | | | | | | | | | | |

Note:
Statistical significance in treatment group versus vehicle group were calculated using the t-test.

### Tumor Weight of Mice at the End of the Experiment

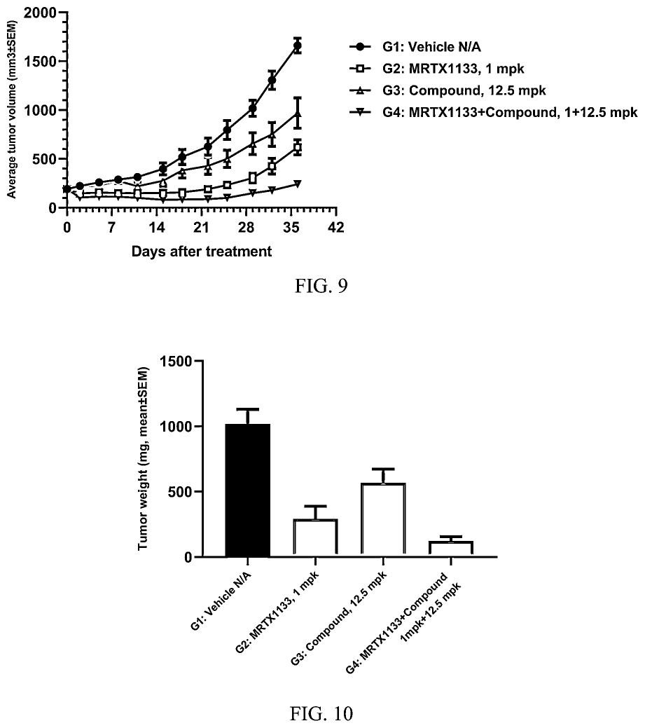

Tumor weight for each group at the end of the study was shown in FIG. 10. The results of the significance (Table 13) analysis showed that compared with the control group, all treatment groups presented significant difference in tumor weight (p<0.05), which is consistent with the results of tumor volume.

TABLE 13
Endpoint tumor weight significance results

| | P value vs. |
|---|---|
| Treatment | control group |
| G2: MRTX1133, 1 mpk, i.p. bid | 0.0021 |
| G3: The compound, 12.5 mpk, p.o. q.d. | 0.0192 |
| G4: MRTX1133 + The compound, | 0.0001 |
| 1 mpk + 12.5 mpk | |

Note:
Statistical significance in treatment group versus vehicle group were calculated using the t-test.

### Summary and Conclusion

On day 36 of the experiment, the tumor volumes of the control group, MRTX1133 group, The compound group and MRTX1133+ The compound group were 1661.33 mm³, 619.45 mm³, 968.93 mm³ and 241.59 mm³, respectively. The T/C value of MRTX1133 group, The compound group and MRTX1133+ The compound group on day 36 was 37.22%, 58.17% and 14.53%, respectively. Correspondingly, the value of TGI was 70.86%, 47.11% and 96.54%, respectively. Compared with Vehicle group, all treatment groups showed significant anti-tumor efficacy (p<0.05) after 36 days of treatment.

In conclusion, all treatment groups showed significant anti-tumor efficacy in AGS model in NOG mice.

### Example 8: The Compound Enhances the Cell Killing Effects of KRAS G12C Inhibitors in NCI-H358 and NCI-H2122 Cells

The aim of this study was to assess the cell killing effects of five different KRAS G12C inhibitors (RMC-4998/Divarasib/LY3537982/Opnurasib and AMG510) in combination with the compound on NCI-H2122 and NCI-H358 cells in vitro.

TABLE 14
Grouping scheme of cell killing assay

| Group | Test Article |
|---|---|
| 1 | RMC-4998 + ¼IC50 The compound |
| 2 | Divarasib + ¼IC50 The compound |
| 3 | LY3537982 + ¼IC50 The compound |
| 4 | Opnurasib + ¼IC50 The compound |
| 5 | AMG510 + ¼IC50 The compound |

TABLE 15
Grouping scheme of cell killing assay

| Group | Test Article |
|---|---|
| 1 | RMC-4998 + ¼IC50 The compound |
| 2 | LY3537982 + ¼IC50 The compound |
| 5 | AMG510 + ¼IC50 The compound |

### Cell Culture

NCI-H2122 and NCI-H358 cells were recovered and maintained by InxMed (Nanjing) Co., Ltd. The cells were cultured by adherent monolayer in vitro at 37° C. in 5% carbon dioxide (CO₂) with RMPI1640 medium containing 10% fetal bovine serum. The cells were digested with trypsin and subcultured two to three times a week. The cells in the exponential growth phase with approximately 80%-90% confluent were harvested and incubated.

### Cell Incubation

NCI-H2122 and NCI-H358 cells were harvested and quantitated by cell counter after trypsin digestion. According to the counting results, the cells were diluted to 3×10⁴ cells per mL with RPMI1640 medium containing 10% fetal bovine serum, and then incubated in 96-well plates. Each well was incubated with 3000 cells in 0.1 mL cell suspension. After incubating, the cells were cultured at 37° C. in 5% CO₂.

### Article Treatment

After growing for 24 hours, the cells were incubated with different concentrations of the test articles. The test articles were uniformly prepared and subpackaged before. In short, the concentration of articles dissolved with DMSO was 10 mM, and the subpackaged volume was 50 μL per piece which was stored at −20° C. in the dark. One of them was taken out for treatment in this experiment.

### CCK-8 Solution Treatment and Measure

After 120 hours of dosing, add 10 μL CCK-8 solution to each well using multi-channel pipettes, and then the cells were cultured at 37° C. in 5% CO₂. After 4 hours, measure the absorbance at 460 nm.

### Statistical Analysis

Data of Cell Viability Rate were analyzed with GraphPad Prism 8.

Cell Viability Rate=[A(Treated)−A(Blank)]/[A(No treated)−A(Blank)]×100%, wherein A(Treated): Absorbance with cells, CCK-8 solution and drugs, A(Blank): Absorbance with medium and CCK-8 solution, without cells, and A(No treated): Absorbance with cells and CCK-8 solution, without drugs.

### Results

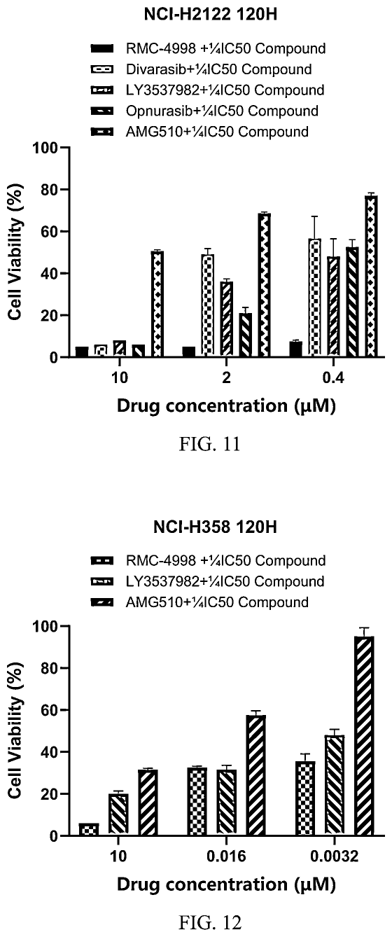

NCI-H2122 cell line result is shown in FIG. 11, and NCI-H358 cell line result is shown in FIG. 12. FIG. 11 shows that the groups of KRAS G12C inhibitors (RMC-4998/Divarasib/LY3537982/Opnurasib) in combination with the compound showed stronger cell-killing effects than AMG510 in combination with the compound. The KRAS G12C inhibitors were tested at three different drug concentrations: 10 μM, 2 μM, and 0.4 μM. The compound was tested at a fixed concentration of ¼ IC₅₀. The compound's IC₅₀ was previously detected on CT26 cells at 23 μM, so the ¼ IC50 The compound in this test was 5.75 μM. FIG. 12 shows that the groups of KRAS G12C inhibitors (RMC-4998/LY3537982) in combination with the compound showed stronger cell-killing effects than AMG510 in combination with the compound. The KRAS G12C inhibitors were tested at three different drug concentrations: 10 μM, 0.016 μM, and 0.0032 μM.

### Conclusion

After 120 hours treatment, the CCK8 assay showed that compared AMG510 in combination with the compound, all other groups of KRAS G12C inhibitors (RMC-4998/Divarasib/LY3537982/Opnurasib) in combination with the compound resulted in significantly greater killing effects.

### Example 9: In Vivo Efficacy Study of MRTX849 and the Compound in NCI-H2122 Xenograft Mouse Model

The aim of this study was to evaluate the in vivo efficacy of MRTX849 and the compound in NCI-H2122 xenograft model in BALB/c nude mice.

### Grouping and Dosing Schedule

TABLE 16
Detailed information of grouping and treatments

| | | | Dose | Dosing | | |
|---|---|---|---|---|---|---|
| | | | level | volume^{b} | Dosing | Dosing |
| Group | Test article | n^{a} | (mg/kg) | (mL/kg) | route | schedule |
| 1 | Vehicle | 5 | N/A | 10 | p.o. | q.d. |
| 2 | MRTX849 | 5 | 30 | 10 | p.o. | q.d. |
| 3 | The compound | 5 | 12.5 | 10 | p.o. | q.d. |
| 4 | MRTX849 + | 5 | 30 + 12.5 | 10 + 10 | p.o. + | q.d. + q.d. |
| | The compound | | | | p.o. | |

Note:
^{a}n: Number of animals per group;
^{b}Dosing volume: 10 mL/kg based on body weight. Animals administration were stopped when body weight loss exceeded 15%, and resumed when body weight returned within 10%.

6- to 8-week-old female Mus musculus BALB/c nude mice, weighted 20-23 g, were purchased from Zhejiang Vital River Laboratory Animal Technology Co., Ltd. The mice were kept in an SPF environment and in Individual Ventilation Cages (5 animals in each cage) at constant temperature (20-26° C.) and humidity (40%-70%). Animals had free access to irradiation sterilized dry granule food and sterile drinking water during the entire study period. The identification labels for each cage contained the following information: number of animals, sex, strain, date received, treatment, study number, group number and the starting date of the treatment. Animals were marked by ear tags.

### Cell Culture

NCI-H2122 tumor cells (Human lung adenocarcinoma cell line, from Medsyin cell bank) were maintained in vitro in RPMI-1640 medium supplemented with 10% FBS and 1% P.S. at 37° C. in an atmosphere of 5% CO₂ in air. The tumor cells were routinely sub-cultured three times weekly. NCI-H2122 cells growing in an exponential growth phase were resuspended in PBS with Matrigel (the volume ratio of PBS: Matrigel=1:1), and adjusted concentration to 5×10⁷/mL for tumor injection.

### Tumor Inoculation and Administration

Each mouse was injected subcutaneously at the right flank with 0.1 mL NCI-H2122 cell suspension (5×10⁶) for tumor development. When the average tumor volume reached 146 mm³, the animals were randomly grouped according to tumor volume on the 6^{th} day after cell injection, followed by drug administration. The day of grouping was defined as day 0. Test article administration and the animal numbers in each group were shown in Table 16.

### Observations

At the time of routine monitoring, the animals were daily checked for any effects of tumor growth and treatments on normal behavior such as mobility, food and water consumption (by visual observation only), eye/hair matting and any other abnormal effects as stated in the protocol. Death and observed clinical signs were recorded on the basis of the numbers of animals within each subset.

### Measurements and Calculations

The major endpoint is to see if the tumor growth can be delayed or mice can be cured. The study is terminated when the average tumor volume of mice in Vehicle group exceeds 2000 mm³ or 3 weeks after administration. Tumor sizes were measured three times weekly in two dimensions using a caliper, and the volume was expressed in mm³ using the formula: V=0.5×a×b² (a, long diameters; b, short diameters). The tumor sizes were then used for the calculations of T/C (%) values using the formula: T/C %=(Tᵢ/T₀)/(Vᵢ/V₀)×100%, where Tᵢ was the average tumor volume of a treatment group on a given day, T₀ was the average tumor volume of the treatment group on the first day of treatment, Vᵢ was the average tumor volume of the control group on the same day with Tᵢ, and V₀ was the average tumor volume of the group on the first day of treatment. Tumor growth inhibition (TGI) was calculated for each group using the formula: TGI (%)=100%−(Tᵢ−T₀)/(Vᵢ−V₀)×100%.

### Statistical Analysis

Summary statistics, including mean and the standard error of the mean (SEM), are provided for the tumor volume and tumor weight of each group at each time point. Statistical analysis of difference in tumor volume versus control group were conducted on the data obtained at each time point.

All data was analyzed with GraphPad Prism 8.0 and the statistical analysis of differences of tumor volume in each group during the whole experiment was conducted by Two-way ANOVA, Fisher's LSD test. One-way ANOVA, Fisher's LSD test were used for tumor weight analysis. P<0.05 was considered to be statistically significant.

### Results

The efficacy study was terminated at day 35 after treatment. Tumor volume and tumor weight during the study were shown as follows.

### Tumor Volume

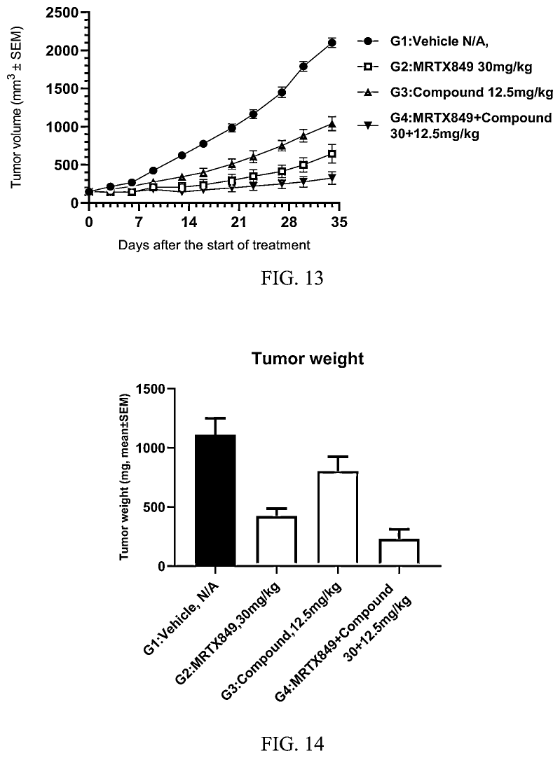

Tumor growth curve of NCI-H2122 tumor-bearing mice after drug administration was shown in FIG. 13. On day 34 of experiment, the tumor volumes of Vehicle group, MRTX849 group, The compound group, MRTX849+ The compound group were 2100.60 mm³, 643.93 mm³, 1039.87 mm³ and 325.54 mm³, respectively.

### T/C (%), TGI (%) and P Values

Relative tumor volume (T/C, %) and tumor growth inhibition (TGI, %) were calculated at each time point and the values were shown in Table 17 and Table 18. Compared with Vehicle group, the T/C values of MRTX849 group, The compound group and MRTX849+ The compound group on day 34 were 30.71%, 49.52% and 15.49%, respectively. Correspondingly, the values of TGI were 74.54%, 54.29% and 90.85%, respectively.

Statistical significance of each group was analyzed and p values were shown in Table 19. Compared with Vehicle group, all treatment groups showed significant difference (p<0.05) in tumor volume. Compared with MRTX849+ The compound group, all other groups showed significant difference (p<0.05) in tumor volume.

TABLE 17
T/C (%) after treatment

| T/C (%) Days after treatment | 0 | 3 | 6 | 9 | 13 | 16 | 20 | 23 | 27 | 30 | 34 |
|---|---|---|---|---|---|---|---|---|---|---|---|
| G2: MRTX849, 30 mpk | — | 67.25 | 52.82 | 48.30 | 32.90 | 30.46 | 30.29 | 29.94 | 28.54 | 27.84 | 30.71 |
| G3: The compound, 12.5 mpk | — | 83.59 | 80.81 | 64.74 | 54.74 | 51.44 | 51.83 | 52.10 | 51.83 | 49.24 | 49.52 |
| G4: MRTX849 + The | — | 66.47 | 53.60 | 42.02 | 22.82 | 21.88 | 20.02 | 18.80 | 17.23 | 15.38 | 15.49 |
| compound 30 mpk + 12.5 mpk | | | | | | | | | | | |

TABLE 18
TGI (%) after treatment

| TGI (%) Days after treatment | 0 | 3 | 6 | 9 | 13 | 16 | 20 | 23 | 27 | 30 | 34 |
|---|---|---|---|---|---|---|---|---|---|---|---|
| G2: MRTX849, 30 mpk | — | 106.09 | 104.26 | 79.17 | 87.79 | 85.83 | 81.99 | 80.21 | 79.56 | 78.65 | 74.54 |
| G3: The compound, 12.5 mpk | — | 53.19 | 42.43 | 53.99 | 59.22 | 59.94 | 56.65 | 54.84 | 53.63 | 55.32 | 54.29 |
| G4: MRTX849 + The | — | 108.62 | 102.55 | 88.73 | 100.95 | 96.40 | 94.03 | 92.93 | 92.11 | 92.18 | 90.85 |
| compound 30 mpk + 12.5 mpk | | | | | | | | | | | |

TABLE 19
P value in each group at specific time point

| Group | 0 | 3 | 6 | 9 | 13 | 16 | 20 | 23 | 27 | 30 | 34 |
|---|---|---|---|---|---|---|---|---|---|---|---|
| G1: Vehicle vs. | 0.9975 | 0.3787 | 0.1109 | 0.0062 | <0.0001 | <0.0001 | <0.0001 | <0.0001 | <0.0001 | <0.0001 | <0.0001 |
| G2: MRTX849 | | | | | | | | | | | |
| G1: Vehicle vs. | 0.9993 | 0.6593 | 0.5155 | 0.0605 | 0.0005 | <0.0001 | <0.0001 | <0.0001 | <0.0001 | <0.0001 | <0.0001 |
| G3: The compound | | | | | | | | | | | |
| G1: Vehicle vs. | 0.9997 | 0.3695 | 0.1176 | 0.0022 | <0.0001 | <0.0001 | <0.0001 | <0.0001 | <0.0001 | <0.0001 | <0.0001 |
| G4: MRTX849 + The | | | | | | | | | | | |
| compound | | | | | | | | | | | |
| G2: MRTX849 vs. | 0.9971 | 0.9862 | 0.9763 | 0.7411 | 0.4308 | 0.405 | 0.2063 | 0.1048 | 0.0408 | 0.0056 | <0.0001 |
| G4: MRTX849 + The | | | | | | | | | | | |
| compound | | | | | | | | | | | |
| G3: The compound vs. | 0.999 | 0.6474 | 0.3583 | 0.2262 | 0.0128 | 0.0043 | 0.0001 | <0.0001 | <0.0001 | <0.0001 | <0.0001 |
| G4: MRTX849 + The | | | | | | | | | | | |
| compound | | | | | | | | | | | |

Note:
Statistical significance in each group were calculated using two-way ANOVA Fisher's LSD test.

### Tumor Weight of Mice at the End of the Experiment

Tumor weight for each group at the end of study was shown in FIG. 14. The results of the significance analysis (Table 20) showed that compared with Vehicle group, MRTX849 and MRTX849+ The compound group showed significant difference (p<0.05). The compound as a single agent showed significant difference (p<0.05) compared with MRTX849+ The compound combination group in tumor weight.

TABLE 20
Endpoint tumor weight significance results

| Group | P Value |
|---|---|
| G1: Vehicle vs. G2: MRTX849 | 0.0003 |
| G1: Vehicle vs. G3: The compound | 0.0551 |
| G1: Vehicle vs. G4: MRTX849 + | <0.0001 |
| The compound | |

Note:
Statistical significance in each group were calculated using one-way ANOVA Fisher's LSD test.

### Conclusion

Compared with Vehicle group, all treatment groups showed anti-tumor efficacy(p<0.05) after 34 days of treatment. Anti-tumor efficacy in MRTX849 and The compound combination group was superior to that in MRTX849 and The compound monotherapy groups.

In conclusion, MRTX849 and the compound administered as a single agent or in combination showed moderate tolerance in the NCI-H2122 xenograft model. Compared with Vehicle group, all treatment groups showed significant anti-tumor efficacy in NCI-H2122 mouse model. Anti-tumor efficacy in combination group of MRTX849 and The compound was superior to that in MRTX849 and The compound monotherapy groups.

### Example 10: In Vivo Efficacy Study of MRTX849 and the Compound in MIA PaCa-2 Xenograft Mouse Model

The aim of this study was to evaluate the in vivo efficacy of MRTX849 and the compound in MIA PaCa-2 xenograft model in BALB/c nude mice.

### Grouping and Dosing Schedule

TABLE 21
Detailed information of grouping and treatments

| | | | Dose | Dosing | | |
|---|---|---|---|---|---|---|
| | | | level | volume^{b} | Dosing | Dosing |
| Group | Test article | n^{a} | (mg/kg) | (mL/kg) | route | schedule |
| 1 | Vehicle | 5 | N/A | 10 | p.o. | q.d. |
| 2 | MRTX849 | 5 | 3 | 10 | p.o. | q.d. |
| 3 | The compound | 5 | 12.5 | 10 | p.o. | q.d. |
| 4 | MRTX849 + | 5 | 3 + 12.5 | 10 + 10 | p.o. + | q.d. + q.d. |
| | The compound | | | | p.o. | |

Note:
^{a}n: Number of animals per group.
^{b}Dosing volume: 10 mL/kg based on body weight. Animals administration were stopped when body weight loss exceeded 15%, and resumed when body weight returned within 10%.

6- to 8-week-old female Mus musculus BALB/c nude mice, weighted 20-23 g, were purchased from Beijing Vital River Laboratory Animal Technology Co., Ltd. The mice were kept in an SPF environment and in Individual Ventilation Cages (5 animals in each cage) at constant temperature (20-26° C.) and humidity (40%-70%). Animals had free access to irradiation sterilized dry granule food and sterile drinking water during the entire study period. The identification labels for each cage contained the following information: number of animals, sex, strain, date received, treatment, study number, group number and the starting date of the treatment. Animals were marked by ear tags.

### Cell Culture

MIA PaCa-2 tumor cells (Human pancreatic cancer cell line, from Medsyin cell bank) were maintained in vitro in DMEM medium supplemented with 10% FBS, 1% P.S. and 2.5% Horse Serum at 37° C. in an atmosphere of 5% CO₂ in air. The tumor cells were routinely sub-cultured three times weekly. MIA PaCa-2 cells growing in an exponential growth phase were resuspended in PBS with Matrigel (the volume ratio of PBS: Matrigel was 1:1), and adjusted concentration to 5×10⁷/mL for tumor injection.

### Tumor Inoculation and Administration

Each mouse was injected subcutaneously at the right flank with 0.1 mL MIA PaCa-2 cell suspension (5×10⁶) for tumor development. When the average tumor volume reached around 157 mm³, the animals were randomly grouped according to tumor volume on the 14^{th} day after cell injection, followed by drug administration. The day of grouping was defined as day 0. Test article administration and the animal numbers in each group were shown in Table 21.

### Observations

At the time of routine monitoring, the animals were daily checked for any effects of tumor growth and treatments on normal behavior such as mobility, food and water consumption (by visual observation only), eye/hair matting and any other abnormal effects as stated in the protocol. Death and observed clinical signs were recorded on the basis of the numbers of animals within each subset.

### Measurements and Calculations

The major endpoint is to see if the tumor growth can be delayed or mice can be cured. The study is terminated when the average tumor volume of mice in Vehicle group exceeds 2000 mm³ or 3 weeks after administration. Tumor sizes were measured twice weekly in two dimensions using a caliper, and the volume was expressed in mm³ using the formula: V=0.5×a×b² (a, long diameters; b, short diameters). The tumor sizes were then used for the calculations of T/C (%) values using the formula: T/C %=(Tᵢ/T₀)/(Vᵢ/V₀)×100%, where Tᵢ was the average tumor volume of a treatment group on a given day, T₀ was the average tumor volume of the treatment group on the first day of treatment, Vᵢ was the average tumor volume of the control group on the same day with Tᵢ, and V₀ was the average tumor volume of the group on the first day of treatment. Tumor growth inhibition (TGI) was calculated for each group using the formula: TGI (%)=100%−(Tᵢ−T₀)/(Vᵢ−V₀)×100%.

### Statistical Analysis

Summary statistics, including mean and the standard error of the mean (SEM), are provided for the tumor volume and tumor weight of each group at each time point. Statistical analysis of difference in tumor volume versus vehicle groups were conducted on the data obtained at each time point.

All data was analyzed with GraphPad Prism 8.0 and the statistical analysis of differences of tumor volume in each group during the whole experiment was conducted by Two-way ANOVA, Fisher's LSD test. One-way ANOVA, Fisher's LSD test were used for tumor weight analysis. P<0.05 was considered to be statistically significant.

### Results

The efficacy study was terminated at day 29 after treatment. Tumor volume during the study were shown as follows.

### Tumor Volume

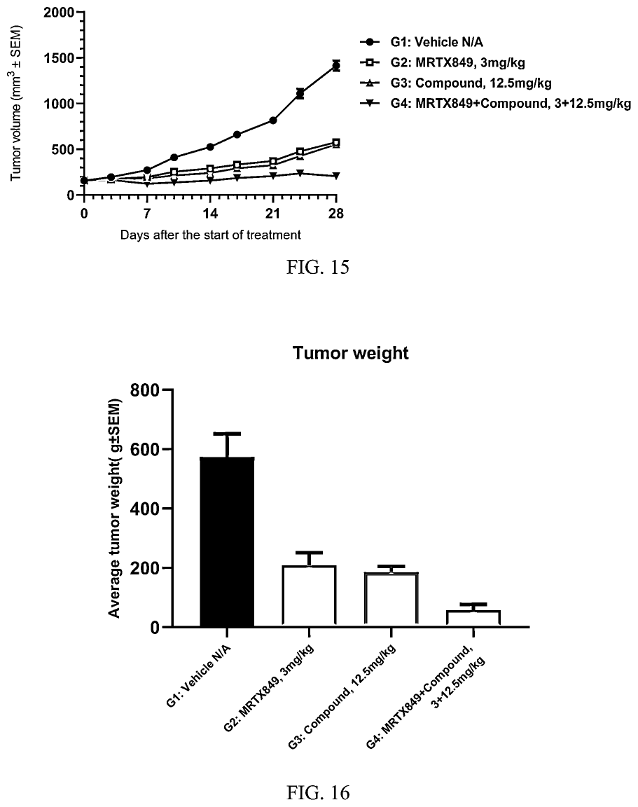

Tumor growth curve of MIA PaCa-2 tumor-bearing mice after drug administration was shown in FIG. 15. On day 28 of experiment, the tumor volumes of Vehicle group, MRTX849 group, The compound group, MRTX849+ The compound group were 1414.49 mm³, 577.35 mm³, 553.74 mm³ and 205.90 mm³, respectively.

### T/C (%), TGI (%) and P Values

Relative tumor volume (T/C, %) and tumor growth inhibition (TGI, %) were calculated at each time point and the values were shown in Table 22 and Table 23. Compared with Vehicle group, the T/C values of MRTX849 group, The compound group and MRTX849+ The compound group on day 28 were 40.85%, 39.14% and 14.57%, respectively. Correspondingly, the values of TGI were 66.59%, 68.48% and 96.15%, respectively.

Statistical significance of each group in tumor volume was analyzed and p values were shown in Table 24. Compared with Vehicle group, all treatment groups showed significant difference (p<0.05) on 28 days after treatment. Compared with MRTX849+ The compound group, all other groups showed significant difference (p<0.05) in tumor volume.

TABLE 22
T/C (%) after treatment

| T/C (%) Days after treatment | 0 | 3 | 7 | 10 | 14 | 17 | 21 | 24 | 28 |
|---|---|---|---|---|---|---|---|---|---|
| G2: MRTX849 | — | 90.21 | 72.08 | 61.87 | 55.19 | 50.27 | 45.73 | 43.16 | 40.85 |
| G3: The compound | — | 86.00 | 66.99 | 52.21 | 45.86 | 44.21 | 39.98 | 38.45 | 39.14 |
| G4: MRTX849 + The | — | 84.53 | 44.87 | 33.39 | 29.91 | 28.05 | 25.44 | 21.33 | 14.57 |
| compound | | | | | | | | | |

TABLE 23
TGI (%) after treatment

| TGI (%) Days after treatment | 0 | 3 | 7 | 10 | 14 | 17 | 21 | 24 | 28 |
|---|---|---|---|---|---|---|---|---|---|
| G2: MRTX849 | — | 50.29 | 66.11 | 61.86 | 63.97 | 65.32 | 67.31 | 66.28 | 66.59 |
| G3: The compound | — | 71.86 | 78.13 | 77.50 | 77.25 | 73.25 | 74.40 | 71.74 | 68.48 |
| G4: MRTX849 + The | — | 79.43 | 130.48 | 108.03 | 100.03 | 94.48 | 92.43 | 91.71 | 96.15 |
| compound | | | | | | | | | |

TABLE 24
P value in each group at specific time point

| P value Days after treatment | 0 | 3 | 7 | 10 | 14 | 17 | 21 | 24 | 28 |
|---|---|---|---|---|---|---|---|---|---|
| G1: Vehicle vs. G2: | 0.9962 | 0.4939 | 0.0075 | <0.0001 | <0.0001 | <0.0001 | <0.0001 | <0.0001 | <0.0001 |
| MRTX849 | | | | | | | | | |
| G1: Vehicle vs. G3: The | 0.9990 | 0.3326 | 0.0017 | <0.0001 | <0.0001 | <0.0001 | <0.0001 | <0.0001 | <0.0001 |
| compound | | | | | | | | | |
| G1: Vehicle vs. | 0.9963 | 0.2817 | <0.0001 | <0.0001 | <0.0001 | <0.0001 | <0.0001 | <0.0001 | <0.0001 |
| G4: MRTX849 + The | | | | | | | | | |
| compound | | | | | | | | | |
| G4: MRTX849 + The | >0.9999 | 0.6937 | 0.0093 | <0.0001 | <0.0001 | <0.0001 | <0.0001 | <0.0001 | <0.0001 |
| compound vs. G2: | | | | | | | | | |
| MRTX849 | | | | | | | | | |
| G4: MRTX849 + The | 0.9952 | 0.9139 | 0.0332 | 0.0066 | 0.0033 | 0.0002 | <0.0001 | <0.0001 | <0.0001 |
| compound vs. G3: The | | | | | | | | | |
| compound | | | | | | | | | |

Note:
Statistical significance in each group were calculated using two-way ANOVA Fisher's LSD test.

### Tumor Weight of Mice at the End of the Experiment

Tumor weight for each group at the end of study was shown in FIG. 16. The results of the significance analysis (Table 25) showed that compared with Vehicle group, all treatment groups showed significant difference (p<0.05). Compared with MRTX849+ The compound group, MRTX849 group showed no significant difference (p>0.05) while The compound group showed significant difference (p<0.05) in tumor weight.

TABLE 25
Endpoint tumor weight significance results

| Groups | P value |
|---|---|
| G1: Vehicle vs. G2: MRTX849 | <0.0001 |
| G1: Vehicle vs. G3: The compound | <0.0001 |
| G1: Vehicle vs. G4: MRTX849 + | <0.0001 |
| The compound | |

Note:
Statistical significance in each group were calculated using one-way Fisher's LSD test.

### Conclusion

MRTX849 and the compound as single drug were well-tolerated in the MIA PaCa-2 xenograft model. Compared with Vehicle group, all treatment groups showed significant anti-tumor efficacy in MIA PaCa-2 mouse model. MRTX849 in combination with the compound present more potential efficacy than MRTX849 and the compound alone.

### Example 11: Evaluation of Anti-Tumor Efficacy of the Compound in the CO-04-0070 Human Colorectal Cancer Patient-Derived Xenograft (PDX) Model in Female BALB/c Nude Mice

The objective of the research was to evaluate the in vivo anti-tumor efficacy of the compound and MRTX849 in the subcutaneous human colorectal cancer PDX model of CO-04-0070 in female BALB/c Nude mice.

### Grouping and Dosing Schedule

TABLE 26
Description of experimental design

| | | | | Dose | | |
|---|---|---|---|---|---|---|
| | | | Dose | Volume | Dose | |
| Group | N^{a} | Treatment^{c} | (mg/kg) | (μL/g)^{b} | Route | Schedule |
| 1 | 4 | Vehicle | — | 10 | PO | QD × 33 |
| | | Control | | | | |
| 2 | 4 | MRTX849 | 10/20 | 10 | PO | QD × 36 |
| 3 | 4 | The compound | 12.5 | 10 | PO | QD × 33 |
| 4 | 4 | MRTX849 + | 10/20 + | 10 + 10 | PO + | QD × 36 + |
| | | The compound | 12.5 | | PO | QD × 36 |

Note:
^{a}N: animal number;
^{b}Dose volume: 10 μL/g based on body weight.
^{c}The dosage of MRTX849 in Group 2 and Group 4 was 10 mg/kg from day 0 to day 4, and 20 mg/kg from day 5 to the end.

### Sample Collection

For Group 1 and Group 3, each tumor was collected for FFPE preparation on day 32 after the start of treatment.

6- to 8-week-old female Mus musculus BALB/c Nude mice, weighted 18-20 g, were purchased from Zhejiang Vital River Laboratory Animal Technology Co., Ltd. The mice were kept in Individual Ventilation Cages (4 animals in each cage) at constant temperature (20-26° C.) and humidity (40%-70%). Animals had free access to irradiation sterilized dry granule food and sterile drinking water during the entire study period. The identification labels for each cage contained the following information: number of animals, sex, strain, date received, treatment, study number, group number and the starting date of the treatment. Animals were marked by ear tags.

### PDX Model

The colorectal cancer PDX model of CO-04-0070 was originally established from a surgically resected clinical sample, which was implanted in BALB/c Nude mice defined as passage 0 (P0). The next passage implanted from P0 tumor was defined as passagel (P1), and so on during continual implantation in mice. The FP5 tumor tissue was used for this study.

### Tumor Inoculation and Animal Grouping

Each mouse was implanted subcutaneously at the right flank with the CO-04-0070 FP5 tumor slices (˜30 mm³) for tumor development. Treatment was started on day 17 after tumor implantation when the average tumor size reached approximately 120 mm³. The animals were assigned into groups using an Excel-based randomization software performing stratified randomization based upon their tumor volumes. Each group consisted of 4 tumor-bearing mice. The test articles were administrated to the mice according to the predetermined regimens as shown in the experimental design table. (Table 26).

### Observations

At the time of routine monitoring, the animals were daily checked for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset.

### Tumor Measurements and Endpoints

The major endpoint was to see if the tumor growth could be delayed or mice could be cured. Tumor size was measured twice weekly in two dimensions using a caliper, and the volume was expressed in mm³ using the formula: V=0.5 a×b² where a and b are the long and short diameters of the tumor, respectively. The tumor sizes were then used for the calculations of both TGI (%) and the T/C (%) values.

TGI was calculated for each group using the formula: TGI (%)=[1−(Ti−T0)/(Vi−V0)]×100; where Ti is the average tumor volume of a treatment group on a given day, T0 is the average tumor volume of the treatment group on the day of treatment starts, Vi is the average tumor volume of the control group on the same day with Ti, and V0 is the average tumor volume of the control group on the day of treatment starts.

The T/C value (in percent) is an indication of antitumor effectiveness; T and C are the mean volumes of the treated and control groups, respectively, on a given day.

Tumor weight was measured at the study termination. T/C_{weight} value (in percent) was calculated using the formula: T/C_{weight} %=T_{weight}/C_{weight}×100% where T_{weight} and C_{weight} were the mean tumor weights of the treated and the control groups, respectively.

### Statistical Analysis

Summary statistics, including mean and the standard error of the mean (SEM), were provided for the tumor volume of each group at each time point.

Statistical analysis of difference in the tumor volume among the groups were conducted on the data obtained at the 32^{nd} day or the 35^{th} day after the start of treatment.

A two-way ANOVA was performed to compare the tumor volume among groups, and comparisons between groups were carried out with Fisher's LSD test. All data was analyzed with GraphPad Prism, p<0.05 was considered to be statistically significant.

### Results

### Mortality, Morbidity, and Body Weight

Animal body weight was monitored regularly as an indicator of toxicity. There were no deaths and no morbidity. All mice did not show obvious body weight loss.

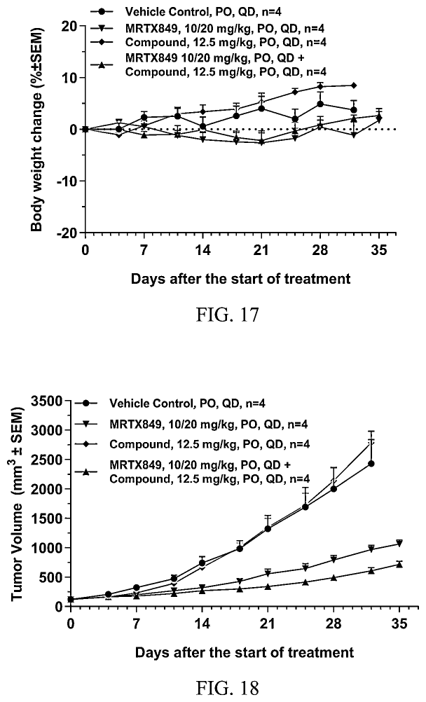

The body weight changes of different treatment groups were shown in FIG. 17.

### Tumor Volume

Mean tumor volumes over time in female BALB/c Nude mice bearing CO-04-0070 xenografts tumor dosed with the compound and MRTX849 were shown in Table.

TABLE 27
Tumor volumes over time

| | Tumor size (mm³)^{a} | | | |
|---|---|---|---|---|
| | | | | MRTX849 + The |
| | | | | compound |
| | | MRTX849 | The compound | 10/20 mg/kg + |
| Days | Vehicle | 10/20 mg/kg | 12.5 mg/kg | 12.5 mg/kg |
| 0 | 119 ± 13 | 119 ± 7 | 120 ± 4 | 120 ± 7 |
| 4 | 205 ± 15 | 161 ± 15 | 162 ± 9 | 164 ± 14 |
| 7 | 322 ± 35 | 199 ± 22 | 229 ± 12 | 177 ± 13 |
| 11 | 473 ± 59 | 268 ± 42 | 392 ± 20 | 220 ± 24 |
| 14 | 741 ± 104 | 319 ± 30 | 665 ± 13 | 267 ± 10 |
| 18 | 981 ± 138 | 426 ± 48 | 1,003 ± 76 | 298 ± 9 |
| 21 | 1,320 ± 229 | 556 ± 79 | 1,346 ± 151 | 340 ± 12 |
| 25 | 1,692 ± 329 | 643 ± 86 | 1,722 ± 204 | 412 ± 7 |
| 28 | 2,000 ± 361 | 791 ± 80 | 2,139 ± 227 | 491 ± 24 |
| 32^{b} | 2,430 ± 408 | 969 ± 70 | 2,785 ± 196 | 606 ± 53 |
| 35 | — | 1,063 ± 67 | — | 714 ± 55 |

Note:
^{a}Mean ± SEM.
^{b}The mice in Group 1 and Group 3 were sacrificed on day 32 after the start of treatment.

### Tumor Growth Inhibition Analysis

TABLE 28
Tumor growth inhibition calculation for the compound
and MRTX849 in the CO-04-0070 PDX model
calculated based on tumor volume measurements at day 32

| | Tumor | | | |
|---|---|---|---|---|
| | Size (mm³)^{a} | T/C^{b} | TGI^{c} | |
| Treatment | at day 32 | (%) | (%) | p value^{d} |
| Vehicle | 2,430 ± 408 | — | — | — |
| MRTX849, 10/20 mg/kg | 969 ± 70 | 39.87 | 63.24 | <0.001 |
| The compound, 12.5 mg/kg | 2,785 ± 196 | 114.65 | −15.40 | 0.030 |
| MRTX849, 10/20 mg/kg + | 606 ± 53 | 24.95 | 78.94 | <0.001 |
| The compound, 12.5 mg/kg | | | | |

Note:
^{a}Mean ± SEM;
^{b}Tumor Growth Inhibition was calculated by dividing the group average tumor volume for the treated group by the group average tumor volume for the control group (T/C);
^{c}TGI (%) = [1 − (T₃₂ − T₀)/(V₃₂ − V₀)] × 100);
^{d}p value was calculated based on tumor size.

TABLE 29
Tumor growth inhibition calculation for the compound
and MRTX849 in the CO-04-0070 PDX model
calculated based on tumor volume measurements at day 35

| | Tumor | |
|---|---|---|
| | Size (mm³)^{a} | |
| Treatment | at day 35 | p value^{b} |
| MRTX849, 10/20 mg/kg | 1,063 ± 67 | — |
| MRTX849, 10/20 mg/kg + | 714 ± 55 | <0.001 |
| The compound, 12.5 mg/kg | | |

Note:
^{a}Mean ± SEM;
^{b}p value was calculated based on tumor size.

### Tumor Growth Curves

Tumor growth curves were shown in FIG. 18.

### Tumor Weights

Tumor weights of different treatment groups of the tumor-bearing mice were shown in Table 30.

TABLE 30
Tumor weights of different treatment groups

| | Tumor |
|---|---|
| | weight (g)^{a} |
| Treatment | at day 32/36 |
| Vehicle | 2.628 ± 0.433 |
| MRTX849, 10/20 mg/kg | 1.194 ± 0.111 |
| The compound, 12.5 mg/kg | 2.772 ± 0.159 |
| MRTX849, 10/20 mg/kg + | 0.747 ± 0.126 |
| The compound, 12.5 mg/kg | |

Note:
^{a}Mean ± SEM.

### Summary and Discussion

Compared with the single treatment group of MRTX849 (10/20 mg/kg, QD), combined treatment with MRTX849 (10/20 mg/kg, QD)+The compound (12.5 mg/kg, QD) produced significant antitumor activity (p<0.001). The efficacy data indicated that the compound synergizes with MRTX849 in the treatment of cancer.

Test compound the compound and MRTX849 at dose levels were tolerated well by the tumor-bearing mice in this study. No obvious body weight loss was observed in all single and combined treatment groups.

The contents of all references (including literature references, issued patents, published patent applications, and co-pending patent applications) cited throughout this application are expressly incorporated herein by reference in their entirety. Unless otherwise defined, all technical and scientific terms used herein have the same meaning as commonly known to those skilled in the art. All features disclosed in this specification may be combined in any combination.

Each feature disclosed in this specification may be replaced by alternative features serving the same, equivalent or similar purpose. Thus, unless expressly stated otherwise, each feature disclosed is only an example of a series of equivalent or similar features.

From the above description, one skilled in the art can easily ascertain the essential characteristics of this invention, and can make various changes and modifications of the invention without departing from the spirit and scope of the invention to adapt them to various usages and conditions. Accordingly, other embodiments are within the scope of the appended claims.

What is claimed is:

1. A method of treating a tumor comprising administering to a subject:
(i) an effective amount of a compound or a pharmaceutically acceptable salt thereof, wherein the compound has a structure of: 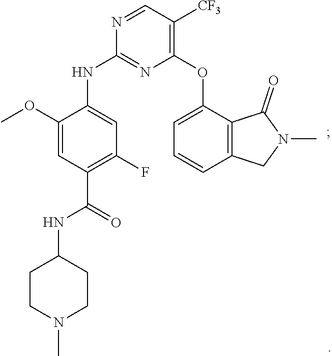
and
(ii) an effective amount of a KRAS inhibitor or a pharmaceutically acceptable salt thereof.

2. The method of claim 1, wherein the tumor is pancreatic cancer, colorectal cancer, lung cancer, kidney cancer, gastric cancer, prostate cancer or, ovarian cancer.

3. The method of claim 2, wherein the tumor is lung adenocarcinoma, non-small cell lung cancer, or gastric adenocarcinoma cancer.

4. The method of claim 1, wherein the tumor has a KRAS mutation of G12A, G12C, G12D, G12R, G12S, G12V, G13C, G13D, G13V, Q61K, Q61L, Q61R, or Q61H.

5. The method of claim 4, wherein the KRAS mutation is a G12C or G12D mutation.

6. The method of claim 1, wherein the tumor is pancreatic cancer with a KRAS G12C mutation, or pancreatic cancer with a KRAS G12D mutation.

7. The method of claim 1, wherein the tumor is gastric cancer with a KRAS G12D mutation.

8. The method of claim 1, wherein the tumor is colorectal cancer with a KRAS G12C mutation, or lung cancer with a KRAS G12C mutation.

9. The method of claim 1, wherein the KRAS inhibitor is BI 1701963, JNJ-74699157, MRTX1257, MRTX849, AMG510, D1553, MRTX1133, RMC-4998, Divarasib, LY3537982, Opnurasib, or a pharmaceutically acceptable salt thereof.

10. The method of claim 9, wherein the KRAS inhibitor is D1553 or a pharmaceutically acceptable salt thereof.

11. The method of claim 9, wherein the KRAS inhibitor is MRTX1133 or a pharmaceutically acceptable salt thereof.

12. The method of claim 9, wherein the KRAS inhibitor is MRTX849 or a pharmaceutically acceptable salt thereof.

13. The method of claim 1, wherein the compound or a pharmaceutically acceptable salt thereof of (i) and the KRAS inhibitor or a pharmaceutically acceptable salt thereof of (ii) are administered simultaneously, alternately, or sequentially.

14. The method of claim 1, wherein the pharmaceutically acceptable salt of the compound of (i) is a tartrate salt.

15. The method of claim 1, further comprising a step of:
a) assessing whether a tissue sample obtained from the subject's cancer has a KRAS mutation before administration.

16. The method of claim 15, further including
c) if the cancer is not characterized by a KRAS mutation, then an anticancer agent other than FAK inhibitors should be administered.

17. A pharmaceutical composition comprising a compound or a pharmaceutically acceptable salt thereof, and a KRAS inhibitor or a pharmaceutically acceptable salt thereof, wherein the compound has a structure of:

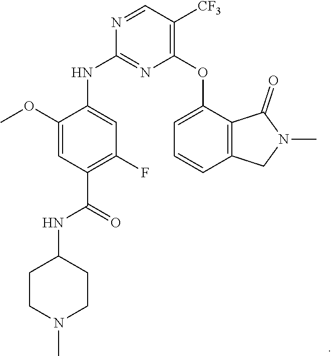

18. The composition of claim 17, wherein the KRAS inhibitor is BI 1701963, MRTX849, AMG510, D1553, MRTX1133, RMC-4998, Divarasib, LY3537982, Opnurasib, or a pharmaceutically acceptable salt thereof.

19. The composition of claim 18, wherein the KRAS inhibitor is D1553 or a pharmaceutically acceptable salt thereof.

20. The composition of claim 18, wherein the KRAS inhibitor is MRTX1133 or a pharmaceutically acceptable salt thereof.

21. The composition of claim 18, wherein the KRAS inhibitor is MRTX849 or a pharmaceutically acceptable salt thereof.

22. The composition of claim 17, wherein the pharmaceutically acceptable salt of the compound of (i) is a tartrate salt.

23. The composition of claim 17, wherein the compound or the pharmaceutically acceptable salt thereof, and the KRAS inhibitor or a pharmaceutically acceptable salt thereof, are present as separate unit doses in the pharmaceutical composition.

24. The composition of claim 17, wherein the compound or the pharmaceutically acceptable salt thereof, and the KRAS inhibitor or a pharmaceutically acceptable salt thereof, are present as a single unit dose in the pharmaceutical composition.

25. A combination of pharmaceutical compositions comprising a first pharmaceutical composition comprising a compound or a pharmaceutically acceptable salt thereof, and a second pharmaceutical composition comprising a KRAS inhibitor or a pharmaceutically acceptable salt thereof, wherein the compound has a structure of:

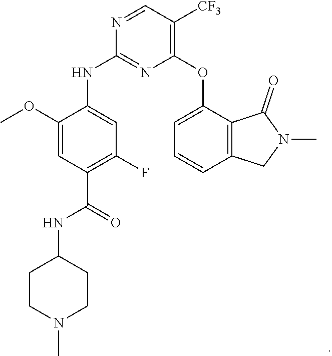

26. The combination of claim 25, wherein the KRAS inhibitor in the second pharmaceutical composition is BI 1701963, MRTX849, AMG510, D1553, MRTX1133, RMC-4998, Divarasib, LY3537982, Opnurasib, or a pharmaceutically acceptable salt thereof.

27. The combination of claim 25, wherein the KRAS inhibitor in the second pharmaceutical composition is D1553 or a pharmaceutically acceptable salt thereof.

28. The combination of claim 25, wherein the KRAS inhibitor in the second pharmaceutical composition is MRTX1133 or a pharmaceutically acceptable salt thereof.

29. The combination of claim 25, wherein the KRAS inhibitor in the second pharmaceutical composition is MRTX849 or a pharmaceutically acceptable salt thereof.

30. The combination of claim 25, wherein the pharmaceutically acceptable salt of the compound in the first pharmaceutical composition is a tartrate salt.
